(19) United States
(12) Patent Application Publication
(10) Pub. No.: US 2023/0303486 A1
(43) Pub. Date: Sep. 28, 2023

(54) CYCLOBUTYL-UREA DERIVATIVES

(71) Applicant: Idorsia Pharmaceuticals Ltd, Allschwil (CH)

(72) Inventors: Olivier BEZENCON, Allschwil (CH); Caroline DEYMIER, Allschwil (CH); Jens-Uwe PETERS, Allschwil (CH); Romain SIEGRIST, Allschwil (CH); Jean-Philippe SURIVET, Allschwil (CH)

(21) Appl. No.: 18/003,226

(22) PCT Filed: Jun. 24, 2021
(86) PCT No.: PCT/EP2021/067288

§ 371 Date: Dec. 23, 2022

(30) Foreign Application Priority Data
Jun. 25, 2020 (WO) PCT/EP2020/067903

(51) Int. Cl. C07C 275/26; C07D 213/40; C07D 239/26; A61P 25/08

(52) U.S. Cl. CPC C07C 275/26; A61P 25/08; C07D 213/40; C07D 239/26; C07C 2601/04

(57) ABSTRACT

The invention relates to compounds of Formula (I)

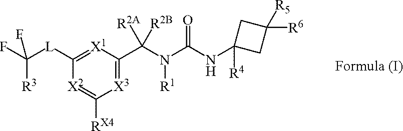

wherein X¹, X², X³, L, R^{X4}, R¹, R^{2A}, R^{2B}, R³, R⁴, R⁵, and R⁶ are as described in the description; to their preparation, to pharmaceutically acceptable salts thereof, to pharmaceutical compositions containing one or more compounds of Formula (I), and to the use of such compounds as medicaments, especially as Kv7 openers.

The present invention relates to cyclobutyl-urea derivatives of Formula (l), and their use as pharmaceuticals. The invention also concerns related aspects including processes for the preparation of the compounds, pharmaceutical compositions containing one or more compounds of Formula (l), and especially their use as Kv7 potassium channel openers. Kᵥ channels are composed of tetramers of α-subunits. Each α-subunit consists of six transmembrane α-helical structures (S1-S6), with an intracellular localization of both the NH₂ and COOH termini. The regions S1-S4 constitute the voltage-sensing domain, whereas the S5 and S6 regions and their linker form the ion-selective pore. Additionally, ancillary proteins are either cytosolic subunits or transmembrane subunits (β).

The Kᵥ7 family comprises five α-subunit Kv7.1-5, encoded by the genes KCNQ1-5. These α-subunits are arranged as homotetramers (Kv7.1, Kv7.2, or Kv7.4) or heterotetramers (Kv7.2/3, Kv7.3/5, or Kv7.4/5). Kv7.1 is mainly localized in cardiomyocytes, gastrointestinal epithelium, skeletal muscles, vascular and non-vascular smooth muscles and the inner ear.

In cardiomyocytes, they slowly activate I_{KS} current which plays a central role in ventricular repolarization. Kv7.2 — Kv7.5 are widely expressed in neuronal tissue with Kv7.2 and Kv7.3 playing a dominant role and found as Kv7.2 homotetramer or Kv7.2/7.3 and Kv7.3/7.5 heterotetramers. They underlie the M-current, which stabilizes the resting membrane potential and reduces action potential firing. Kv7.4 is expressed in outer hair cells, in neurons of the central auditory pathway nuclei, and in dopaminergic neurons of the ventral tegmental area. Kv7.4 and Kv7.5 are both also widely expressed in visceral, vascular and airway smooth muscle, skeletal muscle as Kv7.4 homotetramer or Kv7.4/7.5 heterotetramer. They control auditory physiology and contractility of smooth muscle cells notably. Finally, Kv7.5 is only found in heterotetramers, as discussed previously (Miceli et al. Curr. Med. Chem., 2018, 25, 2637-2660; Jones et al. Handb Exp Pharmacol. 2021).

Activation of the Kᵥ7 channels occurs at potential around -60 mV and results in potassium efflux and membrane hyperpolarization. Dysfunctions or mutations in the Kv7 channels can result physiologically in various channelopathies (C. Bock, A. Link, Future Med. Chem. 2019, 11, 337-355). The neuronal Kᵥ7 channels are responsible for the M-current which regulates neuronal excitability. Due to the dominant role of the M-current in controlling action potential firing, Kv7 openers might be a potential therapy in diseases where enhanced neuronal excitability is a significant aspect of the pathology (Maljevic et al, J. Physiol. 2008, 586(7), 1791-1801; Maljevic et al, Pflugers Arch. 2010, 460(2), 277-88; Jones et al. Handb Exp Pharmacol. 2021), such as epilepsy (Diao et al, 2017, Neuropsychiatry 7(1): 26-31), myokymia (Dedek, Kunath et al. 2001, Proc Natl Acad Sci U S A 98(21): 12272-12277), tinnitus (Li et al. eLife 2015; 4:e07242), neuropathic and inflammatory pain (Rivera-Arconada et al., 2017, Oncotarget 8(8): 12554-12555), substance use disorder such as abuse of alcohol or psychostimulants (Kang et al. 2017, Neuropsychopharmacology 942(9): 1813-1824; Knapp et al. 2014, Am J Drug Alcohol Abuse 40(3): 244-50; McGuier et al. 2016. Addict Biol. 21(6): 1097-1112), psychiatric disorders such as anxiety (Costi et al. 2021, Am J Psychiatry 187(5): 437-446; Hansen et al. 2008, J Physiol 586(7): 1823-32; Kang et al 2017, Neuropsychopharmacology 942(9): 1813-1824; Tan et al. 2020, Mol Psychiatry 25(6): 1323-1333), schizophrenia (Hansen et al. 2008, J Physiol 586(7): 1823-32), depression (Costi et al. 2021, Am J Psychiatry 187(5): 437-446; Friedman et al. 2016, Nat Commun 7: 11671; Su et al 2019, Front Cell Neurosci 13: 557; Tan et al. 2020, Mol Psychiatry 25(6): 1323-1333; Su et al, 2019, Front Cell Neurosci. 13: 557), mania (Grunnet et al. 2014, Eur J Pharmacol 726: 133-7), attention deficit hyperactivity disorder (Grunnet et al. 2014, Eur J Pharmacol 726: 133-7), autism spectrum disorders (Gilling et al. 2013, Front Genet 4: 54; Guglielmi et al. 2015, Front Cell Neurosci 9: 34) and bipolar disorder (Borsotto et al. 2007, Pharmacogenomics J 7(2): 123-32), neurological disorders such as amyotrophic lateral sclerosis (Dafinca et al. 2020, Stem Cell Reports 14(5): 892-908; Ghezzi et al. 2018, J Physiol 596(13): 2611-2629; Wainger et al. 2014, Cell Rep 7(1): 1-11; Wainger et al. 2021, JAMA Neurol 78(2): 186-196), frontotemporal dementia (Dafinca et al. 2020, Stem Cell Reports 14(5): 892-908), primary lateral sclerosis, pseudobulbar palsy, progressive bulbar palsy, progressive muscular atrophy, multiple sclerosis (Pitt et al. 2000, Nat Med 6(1): 67-70), Alzheimer’s disease (Fernandez-Perez et al. 2020, Sci Rep 10(1): 19606; Ghatak et al. 2019, Elife 8; Otto et al. 2004, Neurology 62(5): 714-8), Parkinson’s disease (Chen et al. 2018, Neurosci Bull 34(2): 341-348; Sander et al 2012, Neuropharmacology 62(2): 1052-61), Huntington’s disease (Burgold et al. 2019, Sci Rep 9(1): 6634), Creuzfeld-Jacob disease (Otto et al 2004, Neurology 62(5): 714-8) and acute ischemic stroke (Gribkoff et al. 2001, Nat Med 7(4): 471-7; Bierbower el. 2015, J Neurosci 35(5): 2101-11). Due to the wide distribution of Kᵥ7 channels in other tissues, Kv7 openers might be also useful in diseases affecting the visceral smooth muscles such as functional dyspepsia, irritable bowel syndrome and overactive bladder, in diseases affecting the vascular smooth muscles such as hypertension and cerebral vasospasm, in diseases affecting the airway smooth muscles such as asthma and chronic obstructive pulmonary disease and in hearing disorder and deafness (Haick and Byron 2016, Pharmacol Ther 165: 14-25; Fosmo and Skraastad 2017, Front Cardiovasc Med 4: 75; Xia et al. 2020. Hear Res 388: 107884).

In addition, Kᵥ7 openers might be a potential therapy in disorders associated with KCNQ2, KCNQ3, KCNQ4, KCNQ5 and disorders associated with mutations in KCNQ2, KCNQ3, KCNQ4, KCNQ5 (Dedek, Kunath et al. 2001, Proc Natl Acad Sci U S A 98(21): 12272-12277; Wuttke, Jurkat-Rott et al. 2007, Neurology 69(22): 2045-2053; Millichap, Park et al. 2016, Neurol Genet 2(5): e96; Allen et al 2020, Eur J Paediatr Neurol 2020;24:105-116; Xia et al. 2020. Hear Res 388: 107884).

More specifically, Kv7 openers are suitable antiepileptics drugs, as demonstrated with the FDA-approved drug retigabine/ezogabine. Retigabine/ezogabine activates the potassium current of the different Kv7 channels by binding near the channel gate leading to a stabilization of the channel open state and to a shift of the voltage-dependence of KCNQ activation to more hyperpolarized potentials (Gunthorpe, Large et al. 2012, Epilepsia 53(3): 412-424). Retigabine/ezogabine reduces seizure activity in various rodent models including acute seizure models, genetic models of enhanced seizure sensitivity such as the audiogenic seizure-sensitive DBA2 mice showing generalized tonic-clonic seizures and models of induced epilepsy such as the rat kindling model presenting with focal onset seizures that propagate to bilateral tonic-clonic seizures (Rostock et al. 1996, Epilepsy Res 23(3): 211-223; Tober et al. 1996, Eur J Pharmacol 303(3): 163-169; De Sarro G, Di Paola EG et al. 2001, Naunyn-Schmiedeberg’s Arch Pharmacol 363: 330-336). In two phase three trials retigabine/ezogabine significantly reduced seizure frequency in patients with drug-resistant focal-onset seizures (Brodie, Lerche et al. 2010, Neurology 75(20): 1817-1824; French, Abou-Khalil et al. 2011, Neurology 76(18): 1555-1563).

Moreover, mutations in KCNQ2 and KCNQ3 were recently identified in patients that had been diagnosed with epileptic encephalopathy, infantile/childhood epilepsy syndrome or neurodevelopmental disorders with epilepsy (Helbig and Tayoun 2016, Mol Syndromol 7(4): 172-181; Heyne, Singh et al. 2018, Nat Genet 50(7): 1048-1053). Knock-in mice carrying a KCNQ2 or KCNQ3 variant known to cause reduction of the wild-type potassium current and identified in patients diagnosed with an early onset epileptic syndromes show spontaneous seizures, reduced seizure thresholds, and seizures that are attenuated by retigabine/ezogabine (Singh, Otto et al. 2008, J Physiol 586(14): 3405-3423; Otto, Singh et al. 2009, Epilepsia 50(7): 1752-1759; Tomonoh, Deshimaru et al. 2014, PLoS One 9(2): e88549; Ihara, Tomonoh et al. 2016, PLoS One 11(2): e0150095; Milh, Roubertoux et al. 2020, Epilepsia, doi: 10.1111/epi.16494).

Therefore, Kᵥ7 opener might be a potential therapy in epilepsy including epilepsy with focal onset seizures with or without impaired awareness, with focal onset seizures with motor or nonmotor onset symptoms and with or without focal seizures that develop into bilateral tonic-clonic seizures. Kᵥ7 opener might be a potential therapy in epilepsy with generalized seizures with motor onset symptoms, as well as epilepsy with unknown seizure onset or epilepsy with traumatic brain injury-induced seizures (Diao et al, 2017, Neuropsychiatry 7(1): 26-31; Vigil, Bozdemir et al. 2019, J Cereb Blood Flow Metab: 271678X19857818).

Kv7 opener might be a potential therapy in neonatal onset epilepsy with or without neurodevelopmental impairment including early onset epileptic encephalopathy such as Othahara syndrome or early infantile epileptic encephalopathy, early myoclonic encephalopathy and epilepsy with suppression-burst pattern, but also including benign or self-limiting familial neonatal epilepsy (Singh, Westenskow et al. 2003, Brain 126(Pt 12): 2726-2737; Weckhuysen, Mandelstam et al. 2012, Ann Neurol 71(1): 15-25; Olson, Kelly et al. 2017, Ann Neurol 81(3): 419-429; Milh, Roubertoux et al. 2020, Epilepsia, doi: 10. 1111 /epi. 16494).

Kv7 opener might be a potential therapy in infantile/childhood epilepsy syndromes including epilepsy with neurodevelopmental impairment, focal epilepsies of childhood and idiopathic epilepsy syndromes (Neubauer et al. 2008, Neurology 71(3): 177-83;, Kato et al. 2013, Epilepsia 54(7): 1282-7; Lesca and Depienne 2015, Rev Neurol (Paris) 171(6-7): 539-57; Heyne et al. 2018, Nat Genet 50(7): 1048-53; Lindy et al. 2018, Epilepsia 59(5): 1062-71).

WO2019/161877 discloses alcohol derivatives which activate the Kᵥ7 potassium channels and are claimed to treat disorders responsive to the activation of Kv7 potassium channels. Different cyclic amides, acetamides and ureas which are useful as potassium channel openers, have been disclosed in EP3366683A1 and WO2018/158256 and pentacyclothienyl and indanyl urea derivatives in EP3567034A1. WO 2020/163268 discloses pyridine-4-yl-methyl urea derivatives as Kv7 potentiators.

In the context of a phenotypic screening program aimed at identifying anticonvulsive compounds, new cyclobutyl-urea derivatives were identified, which were found to act pharmacologically as Kᵥ7 opener and which may be useful for the treatment of diseases which are modulated by the KCNQ potassium channels.

1) In a first embodiment, the present invention relates to compounds of Formula (l)

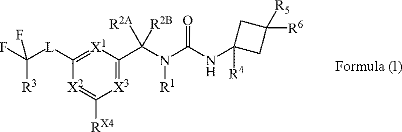

wherein
X¹ represents nitrogen or CR^{x1}; wherein R^{X1} represents hydrogen, halogen (especially fluoro), (C₁₋₄)alkyl, or (C₁₋₄)alkoxy;
X² represents nitrogen or CR^{x2}; wherein R^{x2} represents hydrogen, halogen, (C₁₋₄)alkyl, or (C₁₋₄)alkoxy;
X³ represents nitrogen or CR^{X3}; wherein R^{x3} represents hydrogen, halogen, (C₁₋₄)alkyl, (C₁₋₄)alkoxy, or hydroxy;
R¹ represents hydrogen or methyl;
R^{x4} represents hydrogen, halogen (especially fluoro), or (C₁₋₄)alkyl (especially methyl);
   R^{2A} represents hydrogen; (C₁₋₄)alkyl; (C₂₋₄)alkenyl; (C₂₋₄)alkynyl; (C₃₋₆)cycloalkyl; (C₁₋₄)fluoroalkyl; (C₁₋₄)hydroxyalkyl; (C₁₋₄)alkoxy-(C₁₋₂)alkyl; (C₁₋₂)alkoxy-(C₁₋₂)alkoxy-(C₁₋₂)alkyl; (C₁₋₂)alkyl-S-(C₁₋₂)alkyl; (C₁₋₂)alkyl-(SO₂)-(C₁₋₂)alkyl; cyano; (C₁₋₂)cyanoalkyl; H₂N-C(O)-(C₁₋₂)alkyl; (R^{N1})₂N-(C₁₋₂)alkyl or (R^{N1})₂N—C(O)—, wherein R^{N1} independently represents hydrogen or (C₁₋₂)alkyl; or a 5-membered heteroaryl group containing one to four nitrogen atoms, wherein said 5-membered heteroaryl group is independently unsubstituted or mono-substituted with (C₁₋₄)alkyl; and R^{2B} represents hydrogen or methyl; or
   R^{2A} and R^{2B} form, together with the carbon atom to which they are attached, a ring of 3-to 6 members, wherein the members needed to complete said ring are each independently selected from —CH₂— and —O— and wherein said ring does not contain more than one —O— member;
L represents a direct bond, cycloprop-1,1-diyl, —CHR^{L}—O—*, —O—CH₂—*, —CH₂—NH—*, —CH₂—N(CH₃)—*, —O—, or —(SO₂)—; wherein R^{L} represents hydrogen, (C₁₋₄)alkyl (especially methyl), CH₃—O—CH₂—, or (CH₃)₂NCH₂-; wherein the asterisks indicate the bond which is linked to the aromatic carbon atom;
R³ represents hydrogen or fluoro;
   R⁴ represents hydrogen or (C₁₋₄)alkyl (especially methyl); R⁵ represents hydrogen, fluoro, or hydroxy; and R⁶ represents fluoro or (C₁)fluoroalkyl; or
   R⁴ and R⁵ together represent a bridge selected from —CH₂— and —CH₂CH₂—; and R⁶ represents hydrogen, fluoro, (C₁)fluoroalkyl, or (C₁₋₄)alkyl (especially methyl); and to the salts (in particular pharmaceutically acceptable salts) of such compounds.

Definitions provided herein are intended to apply uniformly to the compounds of Formula (l) (and/or Formula (l_{BC})) as defined in any one of embodiments 1) to 40), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The compounds of Formula (l)(and/or Formula (l_{BC})) as defined in any one of embodiments 1) to 40), may contain one or more stereogenic or asymmetric centers, such as one or more asymmetric carbon atoms. The compounds of Formula (l)(and/or Formula (I_{BC})) may thus be present as mixtures of stereoisomers or in stereoisomerically enriched form, preferably as pure stereoisomers. Mixtures of stereoisomers may be separated in a manner known to a person skilled in the art.

The term “enriched”, for example when used in the context of enantiomers, is understood in the context of the present invention to mean especially that the respective enantiomer is present in a ratio (mutatis mutandis: purity) of at least 70:30, and notably of at least 90:10 (mutatis mutandis: purity of 70% / 90%) with respect to the respective other enantiomer. Preferably the term refers to the respective essentially pure enantiomer. The term “essentially”, for example when used in a term such as “essentially pure” is understood in the context of the present invention to mean especially that the respective stereoisomer / composition / compound etc. consists in an amount of at least 90, especially of at least 95, and notably of at least 99 per cent by weight of the respective pure stereoisomer / composition / compound etc.

Whenever a substituent is denoted as optional, it is understood that such substituent may be absent (i.e. the respective residue is unsubstituted with regard to such optional substituent), in which case all positions having a free valency (to which such optional substituent could have been attached to; such as for example in an aromatic ring the ring carbon atoms and / or the ring nitrogen atoms having a free valency) are substituted with hydrogen where appropriate. Likewise, in case the term “optionally” is used in the context of (ring) heteroatom(s), the term means that either the respective optional heteroatom(s), or the like, are absent (i.e. a certain moiety does not contain heteroatom(s) / is a carbocycle / or the like), or the respective optional heteroatom(s), or the like, are present as explicitly defined.

In this patent application, a dotted line shows the point of attachment of the radical drawn. For example, the radical

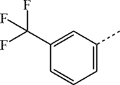

is a 3-(trifluoromethyl)phenyl group.

The term “halogen” means fluorine, chlorine, or bromine, preferably fluorine or chlorine. In case R^{x1}, R^{x2} or R^{X3} represents halogen the term means preferably a fluoro- or chloro-substituent, and more preferably a fluoro-substituent. In case of R^{x4} representing halogen, the term preferably refers to a fluoro-substituent.

The term “alkyl”, used alone or in combination, refers to a straight or branched saturated hydrocarbon chain containing one to four carbon atoms. The term “(C_{x-y})alkyl” (x and y each being an integer), refers to an alkyl group as defined before containing x to y carbon atoms. For example a (C₁₋₄)alkyl group contains from one to four carbon atoms. Representative examples of (C₁₋₄)alkyl groups are methyl, ethyl, n-propyl, iso-propyl, n-butyl, iso-butyl, sec.-butyl and tert.-butyl; preferred is methyl. In case R^{x1}, R^{x2}, R^{x3}, R^{X4}, R^{2A}, R⁴, or R⁶ represents “(C₁₋₄)alkyl” the term means preferably methyl. In case R^{L} represents “(C₁₋₄)alkyl” the term means preferably methyl. In case a “(C₁₋₄)alkyl” is a substituent to a “5-membered heteroaryl group containing one to four nitrogen atoms”, the term means preferably methyl. The term “alkoxy”, used alone or in combination, refers to an alkyl-O- group wherein the alkyl group is as defined before. The term “(C_{x-y})alkoxy” (x and y each being an integer) refers to an alkoxy group as defined before containing x to y carbon atoms. For example a (C₁₋₄)alkoxy group means a group of the formula (C₁₋₄)alkyl-O- in which the term “(C₁₋₄)alkyl” has the previously given significance. Representative examples of (C₁₋₄)alkoxy groups are methoxy, ethoxy, n-propoxy, iso-propoxy, n-butoxy, iso-butoxy, sec.-butoxy and tert.-butoxy. In case R^{X1}, R^{x2}, or R^{X3} represents “(C₁₋₄)alkoxy” the term means preferably methoxy.

The term “(C_{xa-ya})alkoxy-(C_{x-y})alkyl” (x, xa, y and ya each being an integer) refers to an alkyl group as defined before wherein one hydrogen atom has been replaced by (C_{×a-ya})alkoxy as defined before containing xa to ya carbon atoms. In case R^{2A} represents “(C₁₋₄)alkoxy-(C₁₋₂)alkyl” the term means preferably methoxymethyl.

The term “(C_{xa-ya})alkoxy-(C_{x-y})alkoxy” (x, xa, y and ya each being an integer) refers to an alkoxy group as defined before containing x to y carbon atoms wherein one hydrogen atom has been replaced with (C_{xa-ya})alkoxy as defined before containing xa to ya carbon atoms. For example a “(C₁₋₂)alkoxy-(C₁₋₂)alkoxy group” refers to an (C₁₋₂)alkoxy group as defined before containing one or two carbon atoms wherein one hydrogen atom has been replaced with (C₁₋₂)alkoxy as defined before containing one or two carbon atoms. It is preferred that the oxygen-atom of the (C_{x-y})alkoxy group and the oxygen atom of the (C_{xa-ya})alkoxy group are attached to different carbon-atoms of the (C_{x-y})alkoxy group. Representative examples of (C₁₋₂)alkoxy-(C₁₋₂)alkoxy groups include methoxy-methoxy, 2-methoxy-ethoxy, ethoxy-methoxy, and 2-ethoxy-ethoxy.

The term “(C_{xa-ya})alkoxy-(C_{xb-yb})alkoxy-(C_{x-y})alkyl” (x, xa, xb, y, ya and yb each being an integer) refers to an alkyl group as defined before wherein one hydrogen atom has been replaced by (C_{xa-ya})alkoxy-(C_{xb-yb})alkoxy as defined before. In case R^{2A} represents “(C₁₋₂)alkoxy-(C₁₋₂)alkoxy-(C₁₋₂)alkyl” the term means preferably 2-methoxy-ethoxy-methyl.

The term “(C₁₋₄)fluoroalkyl” refers to an alkyl group as defined before containing one to four carbon atoms in which one or more (and possibly all) hydrogen atoms have been replaced with fluorine. The term “(C_{x-y})fluoroalkyl” (x and y each being an integer) refers to a fluoroalkyl group as defined before containing x to y carbon atoms. For example a (C₁₋₄)fluoroalkyl group contains from one to four carbon atoms in which one to nine hydrogen atoms have been replaced with fluorine. Representative examples of (C₁₋₄)fluoroalkyl groups include fluoromethyl, difluoromethyl, trifluoromethyl, 2-fluoroethyl, 2,2-difluoroethyl, and 2,2,2-trifluoroethyl. Preferred are (C₁)fluoroalkyl groups such as fluoromethyl, difluoromethyl, and trifluoromethyl. In case R^{2A} represents “(C₁₋₄)fluoroalkyl” or “(C₁)fluoroalkyl” the term means preferably difluoromethyl or trifluoromethyl, and more preferably difluoromethyl. In case R⁶ represents “(C₁)fluoroalkyl” the term means preferably fluoromethyl, difluoromethyl or trifluoromethyl, and more preferably difluoromethyl or trifluoromethyl.

The term “cycloalkyl”, used alone or in combination, refers to a saturated carbocyclic ring containing three to six carbon atoms. The term “(C_{x-y})cycloalkyl” (x and y each being an integer), refers to a cycloalkyl group as defined before containing x to y carbon atoms. For example a (C₃₋₆)cycloalkyl group contains from three to six carbon atoms. Representative examples of cycloalkyl groups are cyclopropyl, cyclobutyl, cyclopentyl, and cyclohexyl. In case R^{2A} represents “(C₃₋₆)cycloalkyl” the term means preferably cyclopropyl.

The term “alkenyl”, used alone or in combination, refers to a straight or branched hydrocarbon chain containing two to five carbon atoms and one carbon-carbon double bond. The term “(C_{x-y})alkenyl” (x and y each being an integer), refers to an alkenyl group as defined before containing x to y carbon atoms. For example a (C₂-₄)alkenyl group contains from two to four carbon atoms. Representative examples of “(C₂-₄)alkenyl” group are vinyl, prop-1-en-1-yl, prop-2-en-1-yl, but-2-en-1-yl, but-1-en-1-yl, and but-3-en-1-yl. In case R^{2A} represents “(C₂₋₄)alkenyl” the term means preferably prop-2-en-1-yl.

The term “alkynyl”, used alone or in combination, refers to a straight or branched chain hydrocarbon group containing two to six (especially two to four) carbon atoms wherein said hydrocarbon group contains at least one carbon-carbon triple bond. The term “(C_{x-y})alkynyl” (x and y each being an integer), refers to an alkynyl group as defined before, containing x to y carbon atoms. For example a (C₂₋₄)alkynyl group contains from two to four carbon atoms. Representative examples of “(C₂-₄)alkynyl” group are ethynyl, prop-1-yn-1-yl, prop-2-yn-1-yl, but-2-yn-1-yl, but-1-yn-1-yl, and but-3-yn-1-yl.

The term “cyano” refers to a group —CN.

The term “(C_{x-y})cyanoalkyl” (x and y each being an integer) refers to an alkyl group as defined before containing x to y carbon atoms wherein one hydrogen atom has been replaced by a cyano group. Representative examples of “(C₁₋₂)cyanoalkyl” are cyanomethyl and 2-cyanoethyl. In case R^{2A} represents “(C₁₋₂)cyanoalkyl” the term means preferably cyanomethyl.

—(SO₂)— refers to a sulfonyl group and —C(O)— refers to a carbonyl group. In case R^{2A} represents “(C₁₋₂)alkyl-(SO₂)-(C₁₋₂)alkyl” the term means preferably methylsulfonyl-methyl and 2-methylsulfonylethyl.

In case R^{2A} represents “(C₁₋₂)alkyl-S-(C₁₋₂)alkyl” the term means preferably 2-methylthioethyl.

In case R^{2A} represents “H₂N-C(O)-(C₁₋₂)alkyl” the term means preferably 3-amino-3-oxopropyl; “(R^{N1})₂N-(C₁₋₂)alkyl” means preferably dimethylamino-methyl; and “(R^{N1})₂N-C(O)-“ means preferably aminocarbonyl, and methylamino-carbonyl.

In case R^{2A} represents “(C₁₋₄)hydroxyalkyl” the term means preferably hydroxymethyl.

The term “heteroaryl”, used alone or in combination, refers to a heteroaryl-group as specifically defined which group may be unsubstituted or substituted as specifically defined. Representative examples of “5-membered heteroaryl group containing one to four nitrogen atoms” are pyrrolyl, imidazolyl, pyrazolyl, triazolyl, tetrazolyl. Said 5-membered heteroaryl groups are unsubstituted or substituted as explicitly defined.

In case L represents a direct bond, this means that the fragment

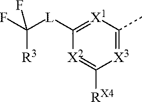

represents:

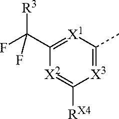

Whenever two substituents together represent a “bridge”, it is to be understood that the atoms to which said substituents are attached, are connected via a —CH₂— or —CH₂CH₂₋bridge as explicitly defined.

2) A second embodiment of the invention relates to compounds of Formula (l) according to embodiment 1), wherein
X¹ represents CR^{x1}; wherein R^{X1} represents hydrogen or halogen (especially fluoro);
X² represents nitrogen or CH;
X³ represents nitrogen or CH;
R¹ represents hydrogen;
R^{x4} represents hydrogen, halogen (especially fluoro), or (C₁₋₄)alkyl (especially methyl);
R^{2A} represents hydrogen; (C₁₋₄)alkyl; (C₁₋₄)fluoroalkyl; (C₁₋₄)hydroxyalkyl; or (C₁₋₄)alkoxy-(C₁₋₂)alkyl;
R^{2B} represents hydrogen;
L represents a direct bond, —CH₂—O—*, or—O—; wherein the asterisk indicates the bond which is linked to the aromatic carbon atom;
R³ represents hydrogen or fluoro;
   R⁴ represents hydrogen or (C₁₋₄)alkyl (especially methyl);
      R⁵ represents hydrogen, fluoro, or hydroxy; and
      R⁶ represents fluoro or (C₁)fluoroalkyl; or
   R⁴ and R⁵ together represent a bridge selected from —CH₂— and —CH₂CH₂—; and
      R⁶ represents hydrogen, fluoro, (C₁)fluoroalkyl, or (C₁₋₄)alkyl (especially methyl); and to the salts (in particular pharmaceutically acceptable salts) of such compounds.

3) Another embodiment of the invention relates to compounds according to any one of embodiments 1) or 2), wherein R^{2A} represents hydrogen, (C₁₋₄)alkyl, (C₁₋₄)fluoroalkyl, (C₁₋₄)hydroxyalkyl, or methoxymethyl (and especially hydrogen, methyl, difluoromethyl, hydroxymethyl, or methoxymethyl); and R^{2B} represents hydrogen; and to the salts (in particular pharmaceutically acceptable salts) of such compounds.

4) Another embodiment of the invention relates to compounds according to any one of embodiments 1) or 2), wherein R^{2A} and R^{2B} both represent hydrogen; and to the salts (in particular pharmaceutically acceptable salts) of such compounds.

5) Another embodiment of the invention relates to compounds according to any one of embodiments 1) to 4), wherein L represents a direct bond or —CH₂—O—*, wherein the asterisk indicates the bond which is linked to the aromatic carbon atom; and to the salts (in particular pharmaceutically acceptable salts) of such compounds.

6) Another embodiment of the invention relates to compounds according to any one of embodiments 1) to 4), wherein L represents a direct bond; and to the salts (in particular pharmaceutically acceptable salts) of such compounds.

7) Another embodiment of the invention relates to compounds according to any one of embodiments 1) to 6), wherein R³ represents hydrogen; and to the salts (in particular pharmaceutically acceptable salts) of such compounds.

8) Another embodiment of the invention relates to compounds according to any one of embodiments 1) to 6), wherein R³ represents fluoro; and to the salts (in particular pharmaceutically acceptable salts) of such compounds.

9) Another embodiment of the invention relates to compounds according to any one of embodiments 1) to 8), wherein R^{x4} represents hydrogen; and to the salts (in particular pharmaceutically acceptable salts) of such compounds.

10) Another embodiment of the invention relates to compounds according to any one of embodiments 1) to 9), wherein R^{X1} represents hydrogen; and to the salts (in particular pharmaceutically acceptable salts) of such compounds.

11) Another embodiment of the invention relates to compounds according to any one of embodiments 1) to 10), wherein R^{X2} represents hydrogen; and to the salts (in particular pharmaceutically acceptable salts) of such compounds.

12) Another embodiment of the invention relates to compounds according to any one of embodiments 1) to 11), wherein R^{X3} represents hydrogen; and to the salts (in particular pharmaceutically acceptable salts) of such compounds.

13) Another embodiment of the invention relates to compounds according to any one of embodiments 1) to 12), wherein X¹ represents CR^{x1}; and to the salts (in particular pharmaceutically acceptable salts) of such compounds.

14) Another embodiment of the invention relates to compounds according to any one of embodiments 1) to 13), wherein X² represents CR^{X2}; and to the salts (in particular pharmaceutically acceptable salts) of such compounds.

15) Another embodiment of the invention relates to compounds according to any one of embodiments 1) to 14), wherein X³ represents CR^{X3}; and to the salts (in particular pharmaceutically acceptable salts) of such compounds.

16) Another embodiment of the invention relates to compounds according to any one of embodiments 1) to 9), wherein each of X¹, X², and X³ represents CH; and to the salts (in particular pharmaceutically acceptable salts) of such compounds.

17) Another embodiment of the invention relates to compounds according to any one of embodiments 1) to 4), wherein the fragment

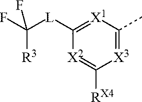

represents:

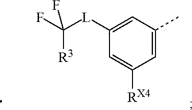

wherein R^{x4} represents hydrogen or halogen (especially fluoro); R³ represents hydrogen or fluoro; and L represents a direct bond, —CH₂—O—*, or—O—; wherein the asterisk indicates the bond which is linked to the aromatic carbon atom; or

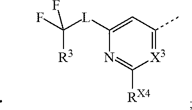

wherein X³ represents nitrogen or CH; R^{x4} represents hydrogen or (C₁₋₄)alkyl (especially methyl); R³ represents hydrogen or fluoro; and L represents —CH₂—O—*, or—O—; wherein the asterisk indicates the bond which is linked to the aromatic carbon atom; and to the salts (in particular pharmaceutically acceptable salts) of such compounds.

18) Another embodiment of the invention relates to compounds according to any one of embodiments 1) to 12), wherein the fragment:

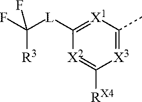

represents:

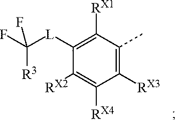

and to the salts (in particular pharmaceutically acceptable salts) of such compounds.

19) Another embodiment of the invention relates to compounds according to any one of embodiments 1) to 9), wherein the fragment:

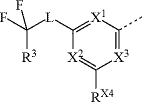

represents:

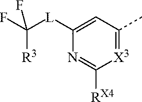

; wherein X³ represents nitrogen or CH; and to the salts (in particular pharmaceutically acceptable salts) of such compounds.

20) Another embodiment of the invention relates to compounds according to any one of embodiments 1) to 4), wherein the fragment:

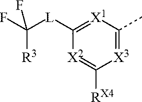

represents a ring independently selected from the following groups A) to C):

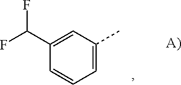

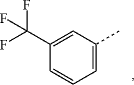

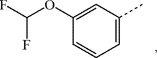

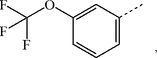

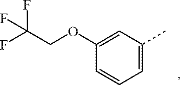

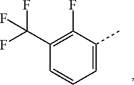

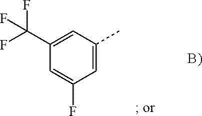

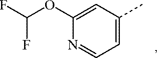

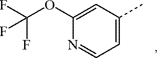

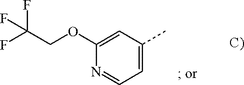

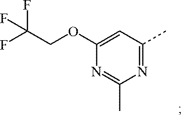

wherein each of the above groups A), B) and C) form a particular sub-embodiment;
and to the salts (in particular pharmaceutically acceptable salts) of such compounds.

21) Another embodiment of the invention relates to compounds of Formula (l)according to any one of embodiments 1) to 3) and 5) to 20), wherein in case R^{2B} represents hydrogen and R^{2A} is different from hydrogen, the carbon atom to which said substituents R^{2A} and R^{2B} are attached is (R)-configurated; and to the salts (in particular pharmaceutically acceptable salts) of such compounds.

22) Another embodiment of the invention relates to compounds of Formula (l)according to any one of embodiments 1) to 3) and 5) to 20), wherein in case R^{2B} represents hydrogen and R^{2A} is different from hydrogen, the carbon atom to which said substituents R^{2A} and R^{2B} are attached is (S)-configurated; and to the salts (in particular pharmaceutically acceptable salts) of such compounds.

23) Another embodiment of the invention relates to compounds according to any one of embodiments 1) to 22), wherein
R⁴ represents hydrogen;
   R⁵ represents hydrogen or fluoro; and
   R⁶ represents fluoro or (C₁)fluoroalkyl (especially fluoro, difluoromethyl or trifluoromethyl); or
R⁴ and R⁵ together represent a —CH₂— bridge; and
   R⁶ represents hydrogen, fluoro, or (C₁)fluoroalkyl (especially difluoromethyl or trifluoromethyl);
   and to the salts (in particular pharmaceutically acceptable salts) of such compounds.

24) Another embodiment of the invention relates to compounds according to any one of embodiments 1) to 22), wherein
R⁴ and R⁵ represent hydrogen; and R⁶ represents (C₁)fluoroalkyl (especially difluoromethyl or trifluoromethyl); or
R⁴ and R⁵ together represent a —CH₂— bridge; and R⁶ represents (C₁)fluoroalkyl (especially difluoromethyl or trifluoromethyl);
and to the salts (in particular pharmaceutically acceptable salts) of such compounds.

25) Another embodiment of the invention relates to compounds according to any one of embodiments 1) to 22), wherein R⁴ represents hydrogen; R⁵ represents hydrogen or fluoro; and R⁶ represents fluoro, difluoromethyl or trifluoromethyl; and to the salts (in particular pharmaceutically acceptable salts) of such compounds.

26) Another embodiment of the invention relates to compounds according to any one of embodiments 1) to 22), wherein R⁴ and R⁵ represent hydrogen; and R⁶ represents difluoromethyl; and to the salts (in particular pharmaceutically acceptable salts) of such compounds.

27) Another embodiment of the invention relates to compounds according to any one of embodiments 1) to 22), wherein R⁴ and R⁵ together represent a —CH₂— bridge; and R⁶ represents hydrogen, fluoro, difluoromethyl, or trifluoromethyl; and to the salts (in particular pharmaceutically acceptable salts) of such compounds.

28) Another embodiment of the invention relates to compounds according to any one of embodiments 1) to 22), wherein R⁴ and R⁵ together represent a —CH₂— bridge; and R⁶ represents difluoromethyl; and to the salts (in particular pharmaceutically acceptable salts) of such compounds.

29) Another embodiment of the invention relates to compounds according to any one of embodiments 1) to 22), wherein the fragment

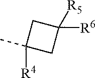

represents a fragment selected from the following groups A) to C):

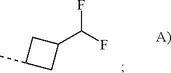

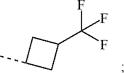

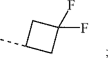

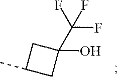

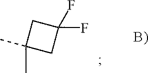

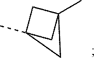

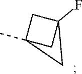

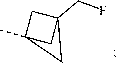

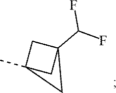

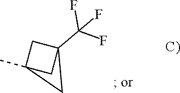

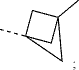

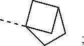

wherein each of the above groups A), B) and C) form a particular sub-embodiment;
and to the salts (in particular pharmaceutically acceptable salts) of such compounds.

30) Another embodiment of the invention relates to compounds according to any one of embodiments 1) to 29), wherein R1 represents hydrogen; and to the salts (in particular pharmaceutically acceptable salts) of such compounds.

31) Another embodiment of the invention relates to compounds according to embodiment 1), that are compounds of Formula (I_{BC})

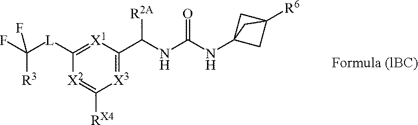

wherein
X¹ represents CR^{x1}; wherein R^{X1} represents hydrogen or halogen (especially fluoro);
X² represents nitrogen or CH;
X³ represents nitrogen or CH;
R^{x4} represents hydrogen, halogen (especially fluoro), or (C₁₋₄)alkyl (especially methyl);
R^{2A} represents hydrogen, (C₁₋₄)alkyl, (C₁₋₄)fluoroalkyl, (C₁₋₄)hydroxyalkyl, or (C₁₋₄)alkoxy-(C₁₋₂)alkyl;
L represents a direct bond, —CH₂—O—*, or —O—;
R³ represents hydrogen or fluoro;
R⁶ represents hydrogen, fluoro, (C₁)fluoroalkyl, or (C₁₋₄)alkyl (especially methyl); and to the salts (in particular pharmaceutically acceptable salts) of such compounds.

32) Another embodiment of the invention relates to compounds according to embodiment 31), wherein X¹ represents CH, X² represents CH, and X³ represents CH; and to the salts (in particular pharmaceutically acceptable salts) of such compounds.

33) Another embodiment of the invention relates to compounds according to any one of embodiments 31) or 32), wherein R^{x4} represents hydrogen; and to the salts (in particular pharmaceutically acceptable salts) of such compounds.

34) Another embodiment of the invention relates to compounds according to any one of embodiments 31) to 33), wherein R^{2A} represents hydrogen, methyl, (C₁)fluoroalkyl, or methoxymethyl; and to the salts (in particular pharmaceutically acceptable salts) of such compounds.

35) Another embodiment of the invention relates to compounds according to any one of embodiments 31) to 33), wherein R^{2A} represents hydrogen; and to the salts (in particular pharmaceutically acceptable salts) of such compounds.

36) Another embodiment of the invention relates to compounds according to any one of embodiments 31) to 35), wherein L represents a direct bond; and to the salts (in particular pharmaceutically acceptable salts) of such compounds.

37) Another embodiment of the invention relates to compounds according to any one of embodiments 31) to 36), wherein R⁶ represents fluoro, (C₁)fluoroalkyl, or methyl; and to the salts (in particular pharmaceutically acceptable salts) of such compounds.

38) Another embodiment of the invention relates to compounds according to any one of embodiments 31) to 36), wherein R⁶ represents difluoromethyl or trifluoromethyl; and to the salts (in particular pharmaceutically acceptable salts) of such compounds.

39) The invention, thus, relates to compounds of the Formula (l) as defined in embodiment 1), and to such compounds further limited by the characteristics of any one of embodiments 2) to 38), under consideration of their respective dependencies; to pharmaceutically acceptable salts thereof; and to the use of such compounds as further described below. For avoidance of doubt, especially the following embodiments relating to the compounds of Formula (l) (and/or Formula (l_{BC})) are thus possible and intended and herewith specifically disclosed in individualized form: 1, 2+1, 3+1, 3+2+1, 4+1, 4+2+1, 5+1, 6+1, 6+2+1, 6+3+1, 6+3+2+1, 6+4+1, 6+4+2+1, 7+1, 8+1, 9+1, 9+2+1, 9+3+1, 9+3+2+1, 9+4+1, 9+4+2+1, 9+5+1, 9+6+1, 9+6+2+1, 9+6+3+1, 9+6+3+2+1, 9+6+4+1, 9+6+4+2+1, 9+7+1, 9+8+1, 10+1, 11+1, 12+1, 13+1, 14+1, 15+1, 16+1, 16+2+1, 16+3+1, 16+3+2+1, 16+4+1, 16+4+2+1, 16+5+1, 16+6+1, 16+6+2+1, 16+6+3+1, 16+6+3+2+1, 16+6+4+1, 16+6+4+2+1, 16+7+1, 16+8+1, 16+9+1, 16+9+2+1, 16+9+3+1, 16+9+3+2+1, 16+9+4+1, 16+9+4+2+1, 16+9+5+1, 16+9+6+1, 16+9+6+2+1, 16+9+6+3+1, 16+9+6+3+2+1, 16+9+6+4+1, 16+9+6+4+2+1, 16+9+7+1, 16+9+8+1, 17+1, 17+2+1, 17+3+1, 17+3+2+1, 17+4+1, 17+4+2+1, 18+1, 19+1, 20+1, 20+2+1, 20+3+1, 20+3+2+1, 20+4+1, 20+4+2+1, 21+1, 22+1, 23+1, 23+2+1, 23+3+1, 23+3+2+1, 23+4+1, 23+4+2+1, 23+5+1, 23+6+1, 23+6+2+1, 23+6+3+1, 23+6+3+2+1, 23+6+4+1, 23+6+4+2+1, 23+7+1, 23+8+1, 23+9+1, 23+9+2+1, 23+9+3+1, 23+9+3+2+1, 23+9+4+1, 23+9+4+2+1, 23+9+5+1, 23+9+6+1, 23+9+6+2+1, 23+9+6+3+1, 23+9+6+3+2+1, 23+9+6+4+1, 23+9+6+4+2+1, 23+9+7+1, 23+9+8+1, 23+10+1, 23+11+1, 23+12+1, 23+13+1, 23+14+1, 23+15+1, 23+16+1, 23+16+2+1, 23+16+3+1, 23+16+3+2+1, 23+16+4+1, 23+16+4+2+1, 23+16+5+1, 23+16+6+1, 23+16+6+2+1, 23+16+6+3+1, 23+16+6+3+2+1, 23+16+6+4+1, 23+16+6+4+2+1, 23+16+7+1, 23+16+8+1, 23+16+9+1, 23+16+9+2+1, 23+16+9+3+1, 23+16+9+3+2+1, 23+16+9+4+1, 23+16+9+4+2+1, 23+16+9+5+1, 23+16+9+6+1, 23+16+9+6+2+1, 23+16+9+6+3+1, 23+16+9+6+3+2+1, 23+16+9+6+4+1, 23+16+9+6+4+2+1, 23+16+9+7+1, 23+16+9+8+1, 23+17+1, 23+17+2+1, 23+17+3+1, 23+17+3+2+1, 23+17+4+1, 23+17+4+2+1, 23+18+1, 23+19+1, 23+20+1, 23+20+2+1, 23+20+3+1, 23+20+3+2+1, 23+20+4+1, 23+20+4+2+1, 23+21+1, 23+22+1, 24+1, 25+1, 25+2+1, 25+3+1, 25+3+2+1, 25+4+1, 25+4+2+1, 25+5+1, 25+6+1, 25+6+2+1, 25+6+3+1, 25+6+3+2+1, 25+6+4+1, 25+6+4+2+1, 25+7+1, 25+8+1, 25+9+1, 25+9+2+1, 25+9+3+1, 25+9+3+2+1, 25+9+4+1, 25+9+4+2+1, 25+9+5+1, 25+9+6+1, 25+9+6+2+1, 25+9+6+3+1, 25+9+6+3+2+1, 25+9+6+4+1, 25+9+6+4+2+1, 25+9+7+1, 25+9+8+1, 25+10+1, 25+11+1, 25+12+1, 25+13+1, 25+14+1, 25+15+1, 25+16+1, 25+16+2+1, 25+16+3+1, 25+16+3+2+1, 25+16+4+1, 25+16+4+2+1, 25+16+5+1, 25+16+6+1, 25+16+6+2+1, 25+16+6+3+1, 25+16+6+3+2+1, 25+16+6+4+1, 25+16+6+4+2+1, 25+16+7+1, 25+16+8+1, 25+16+9+1, 25+16+9+2+1, 25+16+9+3+1, 25+16+9+3+2+1, 25+16+9+4+1, 25+16+9+4+2+1, 25+16+9+5+1, 25+16+9+6+1, 25+16+9+6+2+1, 25+16+9+6+3+1, 25+16+9+6+3+2+1, 25+16+9+6+4+1, 25+16+9+6+4+2+1, 25+16+9+7+1, 25+16+9+8+1, 25+17+1, 25+17+2+1, 25+17+3+1, 25+17+3+2+1, 25+17+4+1, 25+17+4+2+1, 25+18+1, 25+19+1, 25+20+1, 25+20+2+1, 25+20+3+1, 25+20+3+2+1, 25+20+4+1, 25+20+4+2+1, 25+21+1, 25+22+1, 26+1, 27+1, 27+2+1, 27+3+1, 27+3+2+1, 27+4+1, 27+4+2+1, 27+5+1, 27+6+1, 27+6+2+1, 27+6+3+1, 27+6+3+2+1, 27+6+4+1, 27+6+4+2+1, 27+7+1, 27+8+1, 27+9+1, 27+9+2+1, 27+9+3+1, 27+9+3+2+1, 27+9+4+1, 27+9+4+2+1, 27+9+5+1, 27+9+6+1, 27+9+6+2+1, 27+9+6+3+1, 27+9+6+3+2+1, 27+9+6+4+1, 27+9+6+4+2+1, 27+9+7+1, 27+9+8+1, 27+10+1, 27+11+1, 27+12+1, 27+13+1, 27+14+1, 27+15+1, 27+16+1, 27+16+2+1, 27+16+3+1, 27+16+3+2+1, 27+16+4+1, 27+16+4+2+1, 27+16+5+1, 27+16+6+1, 27+16+6+2+1, 27+16+6+3+1, 27+16+6+3+2+1, 27+16+6+4+1, 27+16+6+4+2+1, 27+16+7+1, 27+16+8+1, 27+16+9+1, 27+16+9+2+1, 27+16+9+3+1, 27+16+9+3+2+1, 27+16+9+4+1, 27+16+9+4+2+1, 27+16+9+5+1, 27+16+9+6+1, 27+16+9+6+2+1, 27+16+9+6+3+1, 27+16+9+6+3+2+1, 27+16+9+6+4+1, 27+16+9+6+4+2+1, 27+16+9+7+1, 27+16+9+8+1, 27+17+1, 27+17+2+1, 27+17+3+1, 27+17+3+2+1, 27+17+4+1, 27+17+4+2+1, 27+18+1, 27+19+1, 27+20+1, 27+20+2+1, 27+20+3+1, 27+20+3+2+1, 27+20+4+1, 27+20+4+2+1, 27+21+1, 27+22+1, 28+1, 29+1, 29+2+1, 29+3+1, 29+3+2+1, 29+4+1, 29+4+2+1, 29+5+1, 29+6+1, 29+6+2+1, 29+6+3+1, 29+6+3+2+1, 29+6+4+1, 29+6+4+2+1, 29+7+1, 29+8+1, 29+9+1, 29+9+2+1, 29+9+3+1, 29+9+3+2+1, 29+9+4+1, 29+9+4+2+1, 29+9+5+1, 29+9+6+1, 29+9+6+2+1, 29+9+6+3+1, 29+9+6+3+2+1, 29+9+6+4+1, 29+9+6+4+2+1, 29+9+7+1, 29+9+8+1, 29+10+1, 29+11+1, 29+12+1, 29+13+1, 29+14+1, 29+15+1, 29+16+1, 29+16+2+1, 29+16+3+1, 29+16+3+2+1, 29+16+4+1, 29+16+4+2+1, 29+16+5+1, 29+16+6+1, 29+16+6+2+1, 29+16+6+3+1, 29+16+6+3+2+1, 29+16+6+4+1, 29+16+6+4+2+1, 29+16+7+1, 29+16+8+1, 29+16+9+1, 29+16+9+2+1, 29+16+9+3+1, 29+16+9+3+2+1, 29+16+9+4+1, 29+16+9+4+2+1, 29+16+9+5+1, 29+16+9+6+1, 29+16+9+6+2+1, 29+16+9+6+3+1, 29+16+9+6+3+2+1, 29+16+9+6+4+1, 29+16+9+6+4+2+1, 29+16+9+7+1, 29+16+9+8+1, 29+17+1, 29+17+2+1, 29+17+3+1, 29+17+3+2+1, 29+17+4+1, 29+17+4+2+1, 29+18+1, 29+19+1, 29+20+1, 29+20+2+1, 29+20+3+1, 29+20+3+2+1, 29+20+4+1, 29+20+4+2+1, 29+21+1, 29+22+1, 30+1, 30+2+1, 30+3+1, 30+3+2+1, 30+4+1, 30+4+2+1, 30+5+1, 30+6+1, 30+6+2+1, 30+6+3+1, 30+6+3+2+1, 30+6+4+1, 30+6+4+2+1, 30+7+1, 30+8+1, 30+9+1, 30+9+2+1, 30+9+3+1, 30+9+3+2+1, 30+9+4+1, 30+9+4+2+1, 30+9+5+1, 30+9+6+1, 30+9+6+2+1, 30+9+6+3+1, 30+9+6+3+2+1, 30+9+6+4+1, 30+9+6+4+2+1, 30+9+7+1, 30+9+8+1, 30+10+1, 30+11+1, 30+12+1, 30+13+1, 30+14+1, 30+15+1, 30+16+1, 30+16+2+1, 30+16+3+1, 30+16+3+2+1, 30+16+4+1, 30+16+4+2+1, 30+16+5+1, 30+16+6+1, 30+16+6+2+1, 30+16+6+3+1, 30+16+6+3+2+1, 30+16+6+4+1, 30+16+6+4+2+1, 30+16+7+1, 30+16+8+1, 30+16+9+1, 30+16+9+2+1, 30+16+9+3+1, 30+16+9+3+2+1, 30+16+9+4+1, 30+16+9+4+2+1, 30+16+9+5+1, 30+16+9+6+1, 30+16+9+6+2+1, 30+16+9+6+3+1, 30+16+9+6+3+2+1, 30+16+9+6+4+1, 30+16+9+6+4+2+1, 30+16+9+7+1, 30+16+9+8+1, 30+17+1, 30+17+2+1, 30+17+3+1, 30+17+3+2+1, 30+17+4+1, 30+17+4+2+1, 30+18+1, 30+19+1, 30+20+1, 30+20+2+1, 30+20+3+1, 30+20+3+2+1, 30+20+4+1, 30+20+4+2+1, 30+21+1, 30+22+1, 30+23+1, 30+23+2+1, 30+23+3+1, 30+23+3+2+1, 30+23+4+1, 30+23+4+2+1, 30+23+5+1, 30+23+6+1, 30+23+6+2+1, 30+23+6+3+1, 30+23+6+3+2+1, 30+23+6+4+1, 30+23+6+4+2+1, 30+23+7+1, 30+23+8+1, 30+23+9+1, 30+23+9+2+1, 30+23+9+3+1, 30+23+9+3+2+1, 30+23+9+4+1, 30+23+9+4+2+1, 30+23+9+5+1, 30+23+9+6+1, 30+23+9+6+2+1, 30+23+9+6+3+1, 30+23+9+6+3+2+1, 30+23+9+6+4+1, 30+23+9+6+4+2+1, 30+23+9+7+1, 30+23+9+8+1, 30+23+10+1, 30+23+11+1, 30+23+12+1, 30+23+13+1, 30+23+14+1, 30+23+15+1, 30+23+16+1, 30+23+16+2+1, 30+23+16+3+1, 30+23+16+3+2+1, 30+23+16+4+1, 30+23+16+4+2+1, 30+23+16+5+1, 30+23+16+6+1, 30+23+16+6+2+1, 30+23+16+6+3+1, 30+23+16+6+3+2+1, 30+23+16+6+4+1, 30+23+16+6+4+2+1, 30+23+16+7+1, 30+23+16+8+1, 30+23+16+9+1, 30+23+16+9+2+1, 30+23+16+9+3+1, 30+23+16+9+3+2+1, 30+23+16+9+4+1, 30+23+16+9+4+2+1, 30+23+16+9+5+1, 30+23+16+9+6+1, 30+23+16+9+6+2+1, 30+23+16+9+6+3+1, 30+23+16+9+6+3+2+1, 30+23+16+9+6+4+1, 30+23+16+9+6+4+2+1, 30+23+16+9+7+1, 30+23+16+9+8+1, 30+23+17+1, 30+23+17+2+1, 30+23+17+3+1, 30+23+17+3+2+1, 30+23+17+4+1, 30+23+17+4+2+1, 30+23+18+1, 30+23+19+1, 30+23+20+1, 30+23+20+2+1, 30+23+20+3+1, 30+23+20+3+2+1, 30+23+20+4+1, 30+23+20+4+2+1, 30+23+21+1, 30+23+22+1, 30+24+1, 30+25+1, 30+25+2+1, 30+25+3+1, 30+25+3+2+1, 30+25+4+1, 30+25+4+2+1, 30+25+5+1, 30+25+6+1, 30+25+6+2+1, 30+25+6+3+1, 30+25+6+3+2+1, 30+25+6+4+1, 30+25+6+4+2+1, 30+25+7+1, 30+25+8+1, 30+25+9+1, 30+25+9+2+1, 30+25+9+3+1, 30+25+9+3+2+1, 30+25+9+4+1, 30+25+9+4+2+1, 30+25+9+5+1, 30+25+9+6+1, 30+25+9+6+2+1, 30+25+9+6+3+1, 30+25+9+6+3+2+1, 30+25+9+6+4+1, 30+25+9+6+4+2+1, 30+25+9+7+1, 30+25+9+8+1, 30+25+10+1, 30+25+11+1, 30+25+12+1, 30+25+13+1, 30+25+14+1, 30+25+15+1, 30+25+16+1, 30+25+16+2+1, 30+25+16+3+1, 30+25+16+3+2+1, 30+25+16+4+1, 30+25+16+4+2+1, 30+25+16+5+1, 30+25+16+6+1, 30+25+16+6+2+1, 30+25+16+6+3+1, 30+25+16+6+3+2+1, 30+25+16+6+4+1, 30+25+16+6+4+2+1, 30+25+16+7+1, 30+25+16+8+1, 30+25+16+9+1, 30+25+16+9+2+1, 30+25+16+9+3+1, 30+25+16+9+3+2+1, 30+25+16+9+4+1, 30+25+16+9+4+2+1, 30+25+16+9+5+1, 30+25+16+9+6+1, 30+25+16+9+6+2+1, 30+25+16+9+6+3+1, 30+25+16+9+6+3+2+1, 30+25+16+9+6+4+1, 30+25+16+9+6+4+2+1, 30+25+16+9+7+1, 30+25+16+9+8+1, 30+25+17+1, 30+25+17+2+1, 30+25+17+3+1, 30+25+17+3+2+1, 30+25+17+4+1, 30+25+17+4+2+1, 30+25+18+1, 30+25+19+1, 30+25+20+1, 30+25+20+2+1, 30+25+20+3+1, 30+25+20+3+2+1, 30+25+20+4+1, 30+25+20+4+2+1, 30+25+21+1, 30+25+22+1, 30+26+1, 30+27+1, 30+27+2+1, 30+27+3+1, 30+27+3+2+1, 30+27+4+1, 30+27+4+2+1, 30+27+5+1, 30+27+6+1, 30+27+6+2+1, 30+27+6+3+1, 30+27+6+3+2+1, 30+27+6+4+1, 30+27+6+4+2+1, 30+27+7+1, 30+27+8+1, 30+27+9+1, 30+27+9+2+1, 30+27+9+3+1, 30+27+9+3+2+1, 30+27+9+4+1, 30+27+9+4+2+1, 30+27+9+5+1, 30+27+9+6+1, 30+27+9+6+2+1, 30+27+9+6+3+1, 30+27+9+6+3+2+1, 30+27+9+6+4+1, 30+27+9+6+4+2+1, 30+27+9+7+1, 30+27+9+8+1, 30+27+10+1, 30+27+11+1, 30+27+12+1, 30+27+13+1, 30+27+14+1, 30+27+15+1, 30+27+16+1, 30+27+16+2+1, 30+27+16+3+1, 30+27+16+3+2+1, 30+27+16+4+1, 30+27+16+4+2+1, 30+27+16+5+1, 30+27+16+6+1, 30+27+16+6+2+1, 30+27+16+6+3+1, 30+27+16+6+3+2+1, 30+27+16+6+4+1, 30+27+16+6+4+2+1, 30+27+16+7+1, 30+27+16+8+1, 30+27+16+9+1, 30+27+16+9+2+1, 30+27+16+9+3+1, 30+27+16+9+3+2+1, 30+27+16+9+4+1, 30+27+16+9+4+2+1, 30+27+16+9+5+1, 30+27+16+9+6+1, 30+27+16+9+6+2+1, 30+27+16+9+6+3+1, 30+27+16+9+6+3+2+1, 30+27+16+9+6+4+1, 30+27+16+9+6+4+2+1, 30+27+16+9+7+1, 30+27+16+9+8+1, 30+27+17+1, 30+27+17+2+1, 30+27+17+3+1, 30+27+17+3+2+1, 30+27+17+4+1, 30+27+17+4+2+1, 30+27+18+1, 30+27+19+1, 30+27+20+1, 30+27+20+2+1, 30+27+20+3+1, 30+27+20+3+2+1, 30+27+20+4+1, 30+27+20+4+2+1, 30+27+21+1, 30+27+22+1, 30+28+1, 30+29+1, 30+29+2+1, 30+29+3+1, 30+29+3+2+1, 30+29+4+1, 30+29+4+2+1, 30+29+5+1, 30+29+6+1, 30+29+6+2+1, 30+29+6+3+1, 30+29+6+3+2+1, 30+29+6+4+1, 30+29+6+4+2+1, 30+29+7+1, 30+29+8+1, 30+29+9+1, 30+29+9+2+1, 30+29+9+3+1, 30+29+9+3+2+1, 30+29+9+4+1, 30+29+9+4+2+1, 30+29+9+5+1, 30+29+9+6+1, 30+29+9+6+2+1, 30+29+9+6+3+1, 30+29+9+6+3+2+1, 30+29+9+6+4+1, 30+29+9+6+4+2+1, 30+29+9+7+1, 30+29+9+8+1, 30+29+10+1, 30+29+11+1, 30+29+12+1, 30+29+13+1, 30+29+14+1, 30+29+15+1, 30+29+16+1, 30+29+16+2+1, 30+29+16+3+1, 30+29+16+3+2+1, 30+29+16+4+1, 30+29+16+4+2+1, 30+29+16+5+1, 30+29+16+6+1, 30+29+16+6+2+1, 30+29+16+6+3+1, 30+29+16+6+3+2+1, 30+29+16+6+4+1, 30+29+16+6+4+2+1, 30+29+16+7+1, 30+29+16+8+1, 30+29+16+9+1, 30+29+16+9+2+1, 30+29+16+9+3+1, 30+29+16+9+3+2+1, 30+29+16+9+4+1, 30+29+16+9+4+2+1, 30+29+16+9+5+1, 30+29+16+9+6+1, 30+29+16+9+6+2+1, 30+29+16+9+6+3+1, 30+29+16+9+6+3+2+1, 30+29+16+9+6+4+1, 30+29+16+9+6+4+2+1, 30+29+16+9+7+1, 30+29+16+9+8+1, 30+29+17+1, 30+29+17+2+1, 30+29+17+3+1, 30+29+17+3+2+1, 30+29+17+4+1, 30+29+17+4+2+1, 30+29+18+1, 30+29+19+1, 30+29+20+1, 30+29+20+2+1, 30+29+20+3+1, 30+29+20+3+2+1, 30+29+20+4+1, 30+29+20+4+2+1, 30+29+21+1, 30+29+22+1, 31+1, 32+31+1, 33+31+1, 33+32+31+1, 34+31+1, 34+32+31+1, 34+33+31+1, 34+33+32+31+1, 35+31+1, 35+32+31+1, 35+33+31+1, 35+33+32+31+1, 36+31+1, 36+32+31+1, 36+33+31+1, 36+33+32+31+1, 36+34+31+1, 36+34+32+31+1, 36+34+33+31+1, 36+34+33+32+31+1, 36+35+31+1, 36+35+32+31+1, 36+35+33+31+1, 36+35+33+32+31+1, 37+31+1, 37+32+31+1, 37+33+31+1, 37+33+32+31+1, 37+34+31+1, 37+34+32+31+1, 37+34+33+31+1, 37+34+33+32+31+1, 37+35+31+1, 37+35+32+31+1, 37+35+33+31+1, 37+35+33+32+31+1, 37+36+31+1, 37+36+32+31+1, 37+36+33+31+1, 37+36+33+32+31+1, 37+36+34+31+1, 37+36+34+32+31+1, 37+36+34+33+31+1, 37+36+34+33+32+31+1, 37+36+35+31+1, 37+36+35+32+31+1, 37+36+35+33+31+1, 37+36+35+33+32+31+1, 38+31+1, 38+32+31+1, 38+33+31+1, 38+33+32+31+1, 38+34+31+1, 38+34+32+31+1, 38+34+33+31+1, 38+34+33+32+31+1, 38+35+31+1, 38+35+32+31+1, 38+35+33+31+1, 38+35+33+32+31+1, 38+36+31+1, 38+36+32+31+1, 38+36+33+31+1, 38+36+33+32+31+1, 38+36+34+31+1, 38+36+34+32+31+1, 38+36+34+33+31+1, 38+36+34+33+32+31+1, 38+36+35+31+1, 38+36+35+32+31+1, 38+36+35+33+31+1, 38+36+35+33+32+31+1.

In the list above the numbers refer to the embodiments according to their numbering provided hereinabove whereas “+” indicates the dependency from another embodiment. The different individualized embodiments are separated by commas. In other words, “3+2+1” for example refers to embodiment 3) depending on embodiment 2), depending on embodiment 1), i.e. embodiment “3+2+1” corresponds to the compounds of embodiment 1) further limited by the features of the embodiments 2) and 3).

40) Another embodiment relates to compounds of Formula (l) according to embodiment 1), which are selected from the following compounds:
1-(3,3-Difluoro-cyclobutyl)-3-(3-trifluoromethyl-benzyl)-urea;
1-Bicyclo[1.1.1]pent-1-yl-3-[1-(3-trifluoromethyl-phenyl)-ethyl]-urea;
1-(3-Difluoromethyl-cyclobutyl)-3-[1-(3-trifluoromethyl-phenyl)-ethyl]-urea;
1-(3-Fluoro-bicyclo[1.1.1]pent-1-yl)-3-[1-(3-trifluoromethyl-phenyl)-ethyl]-urea;
1-(3-Difluoromethyl-bicyclo[1.1.1]pent-1-yl)-3-[1-(3-trifluoromethyl-phenyl)-ethyl]-urea;
1-Bicyclo[1.1.1]pent-1-yl-3-[1-(3-trifluoromethoxy-phenyl)-ethyl]-urea;
1-(3-Difluoromethyl-cyclobutyl)-3-[1-(3-trifluoromethoxy-phenyl)-ethyl]-urea;
1-(3-Fluoro-bicyclo[1.1.1]pent-1-yl)-3-[1-(3-trifluoromethoxy-phenyl)-ethyl]-urea;
1-[2,2-Difluoro-1-(3-trifluoromethyl-phenyl)-ethyl]-3-(3-hydroxy-3-trifluoromethyl-cyclobutyl)-urea;
1-(3,3-Difluoro-1-methyl-cyclobutyl)-3-[2,2-difluoro-1-(3-trifluoromethyl-phenyl)-ethyl]-urea;
1-Bicyclo[1.1.1]pent-1-yl-3-[1-(3-difluoromethoxy-phenyl)-ethyl]-urea;
1-(3-Difluoromethyl-bicyclo[1.1.1]pent-1-yl)-3-[2-hydroxy-1-(3-trifluoromethyl-phenyl)-ethyl]-urea;
1-(3-Difluoromethyl-bicyclo[1.1.1]pent-1-yl)-3-[2,2-difluoro-1-(3-trifluoromethyl-phenyl)-ethyl]-urea;
1-(3-Difluoromethyl-bicyclo[1.1.1]pent-1-yl)-3-[2-methoxy-1-(3-trifluoromethyl-phenyl)-ethyl]-urea;
1-(3-Difluoromethyl-bicyclo[1.1.1]pent-1-yl)-3-(2-fluoro-3-trifluoromethyl-benzyl)-urea;
1-(3-Difluoromethyl-bicyclo[1.1.1]pent-1-yl)-3-(3-fluoro-5-trifluoromethyl-benzyl)-urea;
1-(3-Fluoro-bicyclo[1.1.1]pent-1-yl)-3-[3-(2,2,2-trifluoro-ethoxy)-benzyl]-urea;
1-(3-Difluoromethyl-cyclobutyl)-3-[3-(2,2,2-trifluoro-ethoxy)-benzyl]-urea;
1-[3-(2,2,2-Trifluoro-ethoxy)-benzyl]-3-(3-trifluoromethyl-bicyclo[1.1.1]pent-1-yl)-urea;
1-(3-Difluoromethoxy-benzyl)-3-(3-fluoro-bicyclo[1.1.1]pent-1-yl)-urea;
1-(3-Difluoromethoxy-benzyl)-3-(3-difluoromethyl-cyclobutyl)-urea;
1-(3-Difluoromethoxy-benzyl)-3-(3-trifluoromethyl-bicyclo[1.1.1]pent-1-yl)-urea;
1-(3-Trifluoromethoxy-benzyl)-3-(3-trifluoromethyl-bicyclo[1.1.1 ]pent-1-yl)-urea;
1-(3-Fluoro-bicyclo[1.1.1]pent-1-yl)-3-(3-trifluoromethoxy-benzyl)-urea;
1-(3-Difluoromethyl-cyclobutyl)-3-(3-trifluoromethoxy-benzyl)-urea;
1-Bicyclo[1.1.1]pent-1-yl-3-(3-trifluoromethoxy-benzyl)-urea;
1-(3-Difluoromethyl-benzyl)-3-(3-difluoromethyl-cyclobutyl)-urea;
1-(3-Difluoromethyl-benzyl)-3-(3-trifluoromethyl-bicyclo[1.1.1]pent-1-yl)-urea;
1-(3-Difluoromethyl-cyclobutyl)-3-(2-trifluoromethoxy-pyridin-4-ylmethyl)-urea;
1-(2-Trifluoromethoxy-pyridin-4-ylmethyl)-3-(3-trifluoromethyl-bicyclo[1.1.1]pent-1-yl)-urea;
1-(3-Difluoromethyl-cyclobutyl)-3-{2-methoxy-1-[2-(2, 2,2-trifluoro-ethoxy)-pyridin-4-yl]-ethyl}-urea;
1-{2 -M ethoxy-1-[2 -(2,2,2 -trifluoro-ethoxy)-pyrid i n-4-yl]-ethyl}-3-(3-trifl uoromethyl-cyclobutyl)-u rea;
1-[1-(2-Difluoromethoxy-pyridin-4-yl)-ethyl]-3-(3-difluoromethyl-cyclobutyl)-urea;
1-{1-[2-Methyl-6-(2,2,2-trifluoro-ethoxy)-pyrimidin-4-yl]-ethyl}-3-(3-trifluoromethyl-bicyclo[1.1.1]pent-1-yl)-urea;
1-Bicyclo[1.1.1]pent-1-yl-3-(3-trifluoromethyl-benzyl)-urea;
1-(3-Fluoro-bicyclo[1.1.1]pent-1-yl)-3-(3-trifluoromethyl-benzyl)-urea;
1-(3-Trifluoromethyl-benzyl)-3-(3-trifluoromethyl-bicyclo[1.1.1]pent-1-yl)-urea;
1-(3-Difluoromethyl-cyclobutyl)-3-(3-trifluoromethyl-benzyl)-urea;
1-(3-Methyl-bicyclo[1.1.1]pent-1-yl)-3-(3-trifluoromethyl-benzyl)-urea;
1-(3-Fluoromethyl-bicyclo[1.1.1]pent-1-yl)-3-(3-trifluoromethyl-benzyl)-urea;
1-(3-Trifluoromethyl-benzyl)-3-(3-trifluoromethyl-cyclobutyl)-urea;
1-(3-Hydroxy-3-trifluoromethyl-cyclobutyl)-3-(3-trifluoromethyl-benzyl)-urea;
1-Bicyclo[1.1.1]pent-1-yl-3-[2-(2,2,2-trifluoro-ethoxy)-pyridin-4-ylmethyl]-urea;
1-(3-Fluoro-bicyclo[1.1.1]pent-1-yl)-3-[2-(2,2,2-trifluoro-ethoxy)-pyridin-4-ylmethyl]-urea;
1-[2-(2,2,2-Trifluoro-ethoxy)-pyridin-4-ylmethyl]-3-(3-trifluoromethyl-bicyclo[1.1.1]pent-1-yl)-urea;
1-(3-Difluoromethyl-cyclobutyl)-3-[2-(2,2,2-trifluoro-ethoxy)-pyridin-4-ylmethyl]-urea;
1-(3-Difluoromethyl-bicyclo[1.1.1]pent-1-yl)-3-(3-trifluoromethyl-benzyl)-urea;
1-Bicyclo[2.1.1]hex-1-yl-3-(3-trifluoromethyl-benzyl)-urea;
1-(3,3-Difluoro-1-methyl-cyclobutyl)-3-(3-trifluoromethyl-benzyl)-urea;
1-(3-(trifluoromethyl)benzyl)-3-((1s,3s)-3-(trifluoromethyl)cyclobutyl)urea;
1-(3-(trifluoromethyl)benzyl)-3-((1r,3r)-3-(trifluoromethyl)cyclobutyl)urea;
1-((1s,3s)-3-(difluoromethyl)cyclobutyl)-3-(3-(trifluoromethyl)benzyl)urea;
1-((1r,3r)-3-(difluoromethyl)cyclobutyl)-3-(3-(trifluoromethyl)benzyl)urea;
1-{(S)-1-[2-Methyl-6-(2,2,2-trifluoro-ethoxy)-pyrimidin-4-yl]-ethyl}-3-(3-trifluoromethyl-bicyclo[1.1.1]pent-1-yl)-urea;
1-((1r,3r)-3-(difluoromethyl)cyclobutyl)-3-((2-(2,2,2-trifluoroethoxy)pyridin-4-yl)methyl)urea;
1-((1s,3s)-3-(difluoromethyl)cyclobutyl)-3-((2-(2,2,2-trifluoroethoxy)pyridin-4-yl)methyl)urea;
1-((1s,3R)-3-(difluoromethyl)cyclobutyl)-3-((S)-1-(3-(trifluoromethoxy)phenyl)ethyl)urea; and
1-((1r,3S)-3-(difluoromethyl)cyclobutyl)-3-((S)-1-(3-(trifluoromethoxy)phenyl)ethyl)urea;
and to the salts (in particular pharmaceutically acceptable salts) of such compounds.

It is to be understood for any of the above listed compounds, that a stereogenic center, which is not specifically assigned, may be in absolute (R)- or absolute (S)-configuration; for example a compound listed as 1-Bicyclo[1.1.1]pent-1-yl-3-[1-(3-trifluoromethyl-phenyl)-ethyl]-urea may be (S)-1-Bicyclo[1.1.1]pent-1-yl-3-[1-(3-trifluoromethyl-phenyl)-ethyl]-urea, (R)-1-Bicyclo[1.1.1]pent-1-yl-3-[1-(3-trifluoromethyl-phenyl)-ethyl]-urea or any mixture thereof.

Where the plural form is used for compounds, salts, pharmaceutical compositions, diseases or the like, this is intended to mean also a single compound, salt, disease or the like.

Any reference to a compound of Formula (l)(and/or Formula (l_{BC})) as defined in any one of embodiments 1) to 40) is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH, 2008 and ‘Pharmaceutical Salts and Co-crystals’, Johan Wouters and Luc Quéré (Eds.), RSC Publishing, 2012.

The present invention also includes isotopically labelled, especially ²H (deuterium) labelled compounds of Formula (l) (and/or Formula (I_{BC})), which compounds are identical to the compounds of Formula (l) (and/or Formula (l_{BC})) except that one or more atoms have each been replaced by an atom having the same atomic number but an atomic mass different from the atomic mass usually found in nature. Isotopically labelled, especially ²H (deuterium) labelled compounds of Formula (l) (and/or Formula (I_{BC})) and salts thereof are within the scope of the present invention. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of Formula (l) (and/or Formula (I_{BC})) are not isotopically labelled, or they are labelled only with one or more deuterium atoms. In a sub-embodiment, the compounds of Formula (l) (and/or Formula (I_{BC})) are not isotopically labelled at all. Isotopically labelled compounds of Formula (l) (and/or Formula (I_{BC})) may be prepared in analogy to the methods described hereinafter, but using the appropriate isotopic variation of suitable reagents or starting materials.

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

Unless used regarding temperatures, the term “about” (or alternatively “around”) placed before a numerical value “X” refers in the current application to an interval extending from X minus 10% of X to X plus 10% of X, and preferably to an interval extending from X minus 5% of X to X plus 5% of X. In the particular case of temperatures, the term “about” (or alternatively “around”) placed before a temperature “Y” refers in the current application to an interval extending from the temperature Y minus 10° C. to Y plus 10° C., and preferably to an interval extending from Y minus 5° C. to Y plus 5° C. Besides, the term “room temperature” as used herein refers to a temperature of about 25° C.

The compounds of formula (l) (and/or Formula (I_{BC})) as defined in any one of embodiments 1) to 40) and their pharmaceutically acceptable salts can be used as medicaments, e.g. in the form of pharmaceutical compositions for enteral (such as especially oral) or parenteral administration (including topical application or inhalation).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of Formula (l) (and/or Formula (l_{BC})) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention/prophylaxis or treatment of a disease or disorder mentioned herein comprising administering to a subject a pharmaceutically active amount of a compound of Formula (l) (and/or Formula (I_{BC})) as defined in any one of embodiments 1) to 40).

For avoidance of any doubt, if compounds are described as useful for the prevention or treatment of certain diseases, such compounds are likewise suitable for use in the preparation of a medicament for the prevention or treatment of said diseases.

Another aspect of the invention concerns a method for the prevention/prophylaxis or the treatment of a disease or disorder as mentioned below in a patient comprising the administration to said patient of a pharmaceutically active amount of a compound of Formula (l) (and/or Formula (I_{BC})) as defined in any one of embodiments 1) to 40) or a pharmaceutically acceptable salt thereof.

The compounds according to Formula (l) (and/or Formula (I_{BC})) as defined in any one of embodiments 1) to 40) are useful for the prevention/prophylaxis or treatment of diseases or disorders associated with KCNQ2, KCNQ3, KCNQ4, KCNQ5 and/or diseases or disorders associated with mutations in KCNQ2, KCNQ3, KCNQ4, KCNQ5.

Such diseases or disorders associated with KCNQ2, KCNQ3, KCNQ4, KCNQ5 and/or diseases or disorders associated with mutations in KCNQ2, KCNQ3, KCNQ4, KCNQ5 may in particular be defined as comprising epilepsy, myokymia, tinnitus, hearing disorders, neuropathic and inflammatory pain, psychiatric disorders, substance use disorders, neurological disorders, and diseases affecting the smooth muscles (and especially epilepsy, myokymia, tinnitus, neuropathic and inflammatory pain, psychiatric disorders, and diseases affecting the smooth muscles).

Epilepsy may be defined as comprising:
epilepsy with focal onset seizures (with or without impaired awareness, with motor or nonmotor onset symptoms);
epilepsy with generalized seizures with motor onset symptoms;
epilepsy with unknown seizure onset;
epilepsy with traumatic brain injury-induced seizures;
neonatal epilepsy including early onset epileptic encephalopathy with or without neurodevelopmental impairment (such as Othahara syndrome, early infantile epileptic encephalopathy, early myoclonic encephalopathy, epilepsy with suppression-burst pattern, benign or self-limiting familial neonatal epilepsy);
infantile/childhood epilepsy syndromes including epilepsy with neurodevelopmental impairment, focal epilepsies of childhood and idiopathic epilepsy syndromes.

Diseases affecting the smooth muscles may be defined as comprising diseases affecting the visceral smooth muscles (such as functional dyspepsia, irritable bowel syndrome and overactive bladder), diseases affecting the vascular smooth muscles (such as hypertension, and cerebral vasospasm), diseases affecting the airway smooth muscles (such as asthma and chronic obstructive pulmonary disease) and hearing disorders.

Substance use disporders may be defined as comprising abuse of alcohol or psychostimulants.

Psychiatric disorders may be defined as comprising anxiety, schizophrenia, depression, mania, attention deficit hyperactivity disorder, bipolar disorder and autism spectrum disorders (and especially anxiety, schizophrenia, mania, and autism).

Neurological disorders may be defined as comprising diseases caused by changes in neurons and/or motoneurons excitability, (and notably amyotrophic lateral sclerosis, frontotemporal dementia, primary lateral sclerosis, pseudobulbar palsy, progressive bulbar palsy, progressive muscular atrophy, multiple sclerosis, Alzheimer’s disease, Parkinson’s disease, Huntington’s disease, Creutzfeld-Jacob disease, acute ischemic stroke).

Notably, compounds of Formula (l) (and/or Formula (l_{BC})) according to any one of embodiments 1) to 40), or pharmaceutically acceptable salts thereof, are suitable for the prevention/prophylaxis or treatment of neuropathic pain, inflammatory pain, amyotrophic lateral sclerosis, depression, tinnitus and/or epilepsy (especially epilepsy with focal seizures, epilepsy with generalized seizures, epilepsy with unknown onset, neonatal epilepsy, and/or infantile/childhood epilepsy syndromes with or without neurodevelopmental decline).

Especially, compounds of Formula (l) (and/or Formula (I_{BC})) according to any one of embodiments 1) to 40), or pharmaceutically acceptable salts thereof, are suitable for the prevention/prophylaxis or treatment of epilepsy; and especially of epilepsy with focal seizures, epilepsy with generalized seizures, epilepsy with unknown onset, neonatal epilepsy, and/or infantile/childhood epilepsy syndromes with or without neurodevelopmental decline.

### PREPARATION OF COMPOUNDS OF FORMULA (L)

A further aspect of the invention is a process for the preparation of compounds of Formula (I). Compounds according to Formula (l) of the present invention can be prepared from commercially available or well known starting materials according to the methods described in the experimental part; by analogous methods; or according to the general sequence of reactions outlined below, wherein R¹, R^{2A}, R^{2B}, R³, R⁴, R⁵, R⁶, R^{X4}, X¹, X², X³, and L are as defined for Formula (l). Other abbreviations used herein are explicitly defined, or are as defined in the experimental section. In some instances the generic groups R¹, R^{2A}, R^{2B}, R³, R⁴, R^{5,} R⁶, R^{X4}, X¹, X², X³, and L might be incompatible with the assembly illustrated in the schemes below and so will require the use of protecting groups (PG). The use of protecting groups is well known in the art (see for example “Protective Groups in Organic Synthesis”, T.W. Greene, P.G.M. Wuts, Wiley-Interscience, 1999). For the purposes of this discussion, it will be assumed that such protecting groups as necessary are in place. The compounds obtained may also be converted into salts, especially pharmaceutically acceptable salts thereof in a manner known per se.

### General Preparation Routes

Generally, compounds of Formula l can be synthesised by treating an amine of Stucture 2 (or the corresponding salt, like HCl or TFA salts) with an isocyanate 3 in the presence of a base such as NEt₃ or DIPEA in solvent such as DCM or MeCN. Alternatively, an isocyanate of Structure 4 can be reacted with an amine 5 (or the corresponding salt, like HCl or TFA salts) in the presence of a base such as NEt₃ or DIPEA in solvent such as DCM or MeCN to afford compounds of Formula I-A (Scheme 1).

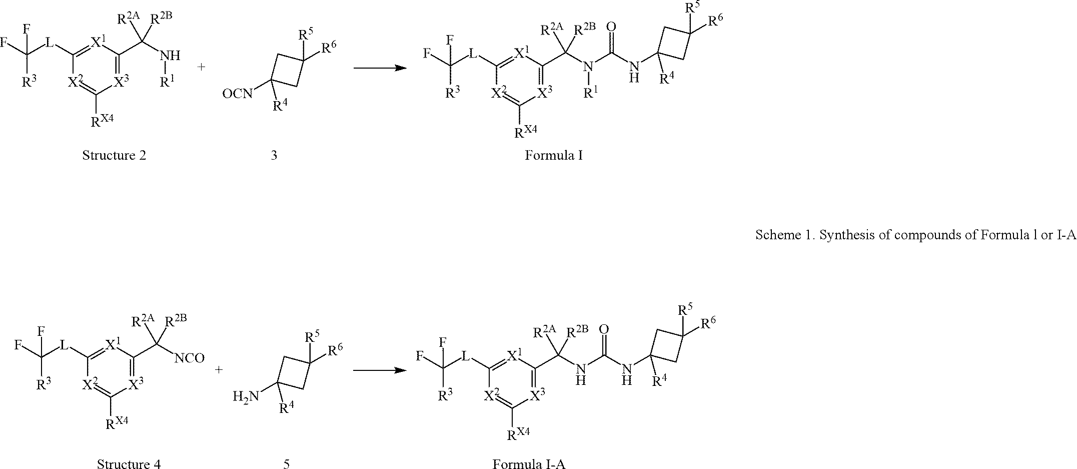

Alternatively, an amine of Structure 2 (or the corresponding salt, like HCl or TFA salts) is condensed with 4-nitrophenyl chloroformate in the presence of a base like NEt₃ or DIPEA to give a carbamate 6 (Scheme 2). The carbamate 6 is then treated with an amine 5 (or the corresponding salt, like HCl or TFA salts) in the presence of a base like NEt₃ in a solvent like THF to yield a compound of Formula l. The sequence can also start by first reacting an amine 5 (or the corresponding salt, like HCl or TFA salts) with 4-nitrophenyl chloroformate in the presence of a base like NEt₃ or DIPEA to give a carbamate 7 (Scheme 2). The carbamate 7 is then treated with an amine of Structure 2 (or the corresponding salt, like HCl or TFA salts) in the presence of a base like NEt₃ in solvent like THF to yield a compound of Formula l.

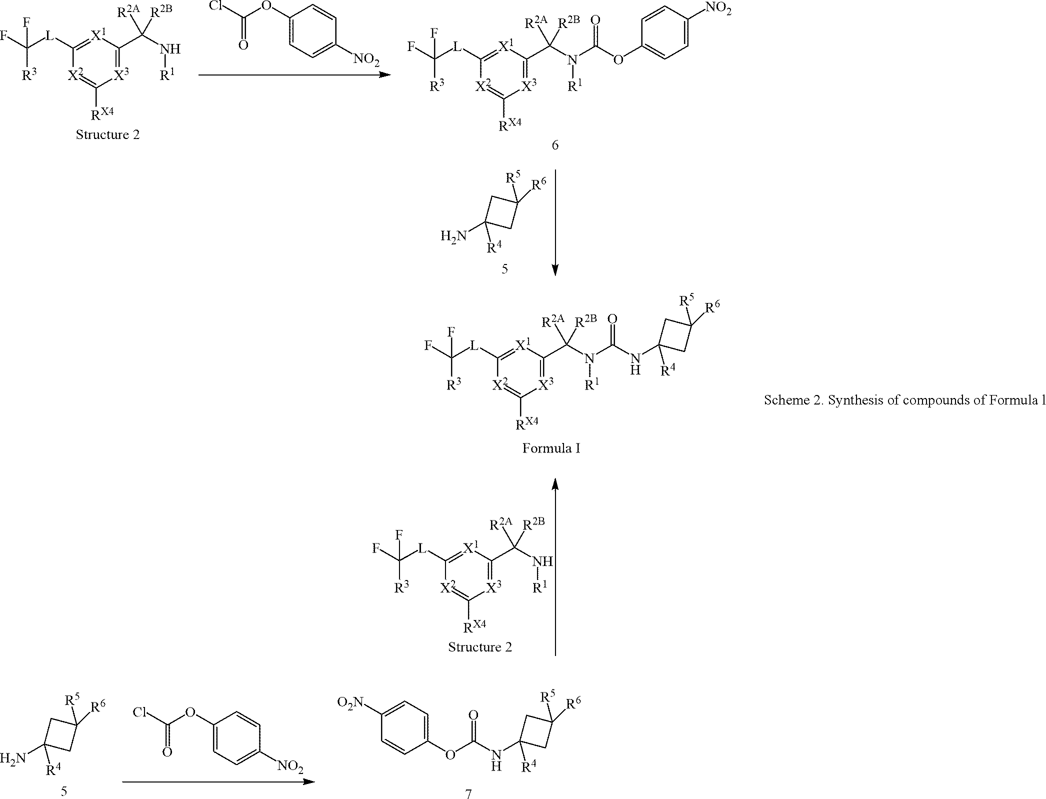

In another aspect, an amine of Structure 2 (or the corresponding salt, like HCl or TFA salts) is activated with a reagent like CDI, triphosgene, or trifluoroethoxycarbonate and the activated intermediate is in-situ treated with an amine 5 (or the corresponding salt, like HCl or TFA salts) to yield a compound of Formula l (Scheme 3). Conversely, an amine 5 (or the corresponding salt, like HCl or TFA salts) can be activated with a reagent like CDI, triphosgene, or trifluoroethoxycarbonate and the activated intermediate is in-situ treated with an amine of Structure 2 (or the corresponding salt, like HCl or TFA salts) to yield a compound of Formula l.

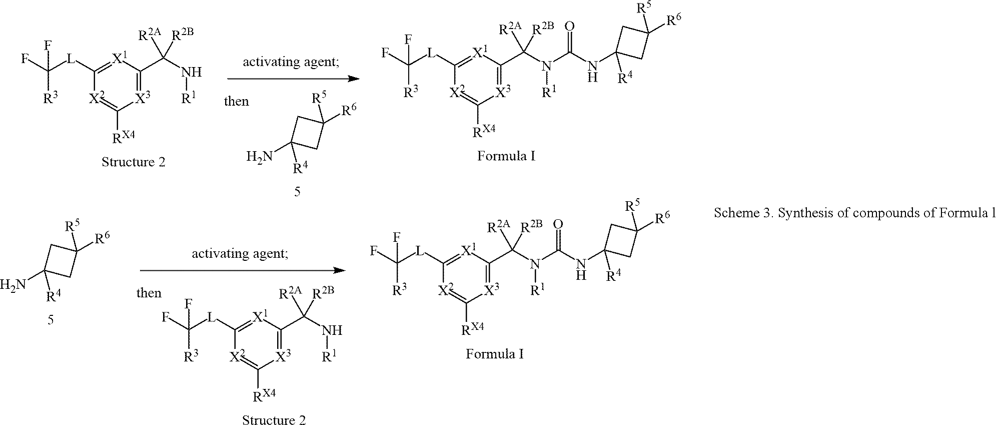

Amines of Structure 2-A or 2-B can be synthesized by taking advantage of the Ellman’s auxiliary (Scheme 4). Thus, an aldehyde 8 is treated with tert-butanesulfinamide 9 in the presence of Ti(OEt)₄ to provide a tert-butanesulfinyl imine 10. Compound 10 is then treated with a nucleophile such as a Grignard reagent 11 to afford a protected amine 12. The tert-butanesulfinyl group is then cleaved under mild acidic conditions like HCl in MeOH to afford an amine of Structure 2-A (or the corresponding HCl salt). Alternatively, imine 10 can be reduced with a reducing agent like NaBH₄ in MeOH to yield a protected amine 13. The tert-butanesulfinyl group is then cleaved under mild acidic conditions like HCl in MeOH to afford an amine of Structure 2-B (or the corresponding HCl salt). Alternatively, a ketone 14 can be reacted with tert-butanesulfinamide 9 in the presence of Ti(OEt)₄ to provide a tert-butanesulfinyl imine 15. Compound 15 is then treated with a Grignard or lithiated reagent 16 to afford a protected amine 17. The tert-butanesulfinyl group is then cleaved under mild acidic conditions like HCl in MeOH to afford an amine of Structure 2-C (or the corresponding HCl salt).

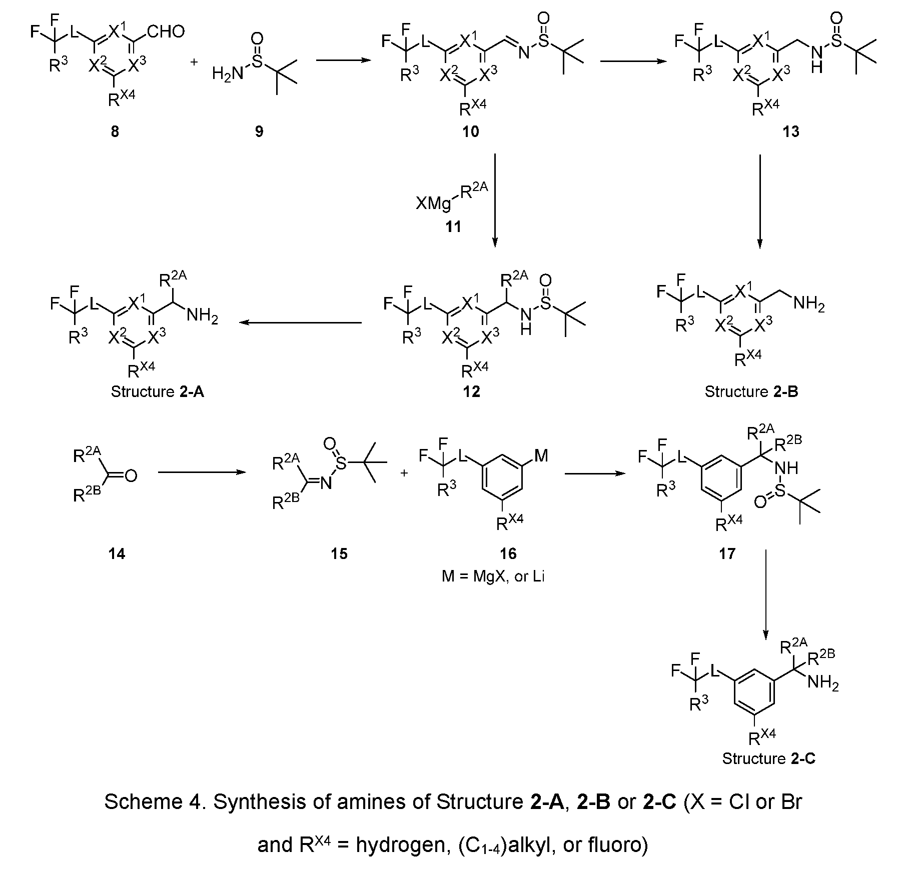

In another aspect, an amine of Structure 2-A can be synthesized using photoredox catalysis (Scheme 5). A bromide 18 is reacted with a Boc-protected amino acid 19 in the presence of an iridium catalyst like [lr{dF(CF₃)ppy}₂(dtbpy)]PF₆ and a nickel catalyst like NiCl₂•glyme in a solvent like DMSO or DMA under blue LED irradiation to give a Boc-protected amine 20 (Science 2014, 345, 437-440). The Boc-protecting group is then cleaved under acidic conditions like TFA in DCM or 4 M HCl in dioxane to give an amine of Structure 2-A (or the corresponding salt, like HCl or TFA salts).

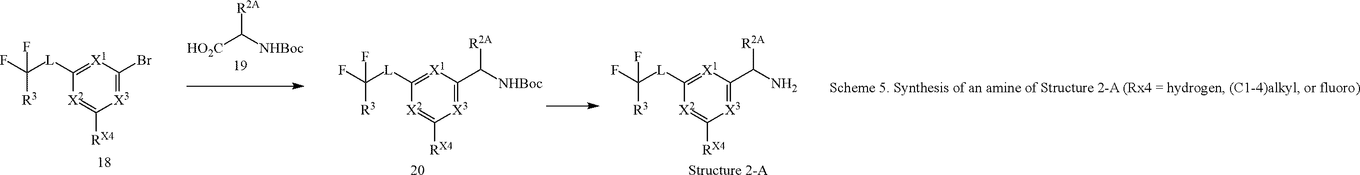

An amine of Structure 2-B can also be obtained from the corresponding nitrile 21 (Scheme 6). A solution of a nitrile 21 in MeOH can be reduced using a catalyst like Ra/Ni under an H₂-atmosphere (in flow or batch mode) or LiAlH₄ in a solvent like THF to give an amine of Structure 2-B. Alternatively, nitrile 21 can be reduced using a nickel catalyst like NiCI₂•6H₂O and NaBH₄ in the presence of Boc₂O to give a Boc-protected amine 22. Deprotection under acidic conditions like TFA in DCM or HCl in dioxane yield an amine of Structure 2-B (or the corresponding HCl or TFA salt). Nitrile 21 can also be converted to the corresponding ketones 23 using MeMgBr in a solvent like Et₂O followed by an aqueous acidic treatment. Ketone 23 can undergo a reductive amination in a solvent like MeOH with for example ammonium acetate and sodium cyanoborohydride to give an amine of Structure 2-A (where R^{2A} is methyl). Moreover, nitrile 21 can be treated first with MeMgBr in a solvent like 2-methyltetrahydrofuran and then with NaBH₄ to give an amine of Structure 2-A (where R^{2A} is methyl). Nitrile 21 can also be subjected to a Kulinkovich reaction in Et₂O using EtMgBr in the presence of a titanium salt like Ti(OiPr)₄ and borontrifluoride to give an amine of Structure 2-D. Finally, nitrile 21 can react with a Boc-protected amino acid in the presence of cesium fluoride and an iridium catalyst like Ir(p-F(t-Bu)-ppy)₃ in a solvent like DMSO or DMA under blue LED irradiation to give a Boc-protected amine 24 (JACS 2014, 136, 5257-5260). The protecting group can then be cleaved under acidic conditions like TFA in DCM or HCl in dioxane to give an amine of Structure 2-E (or the corresponding HCl or TFA salt).

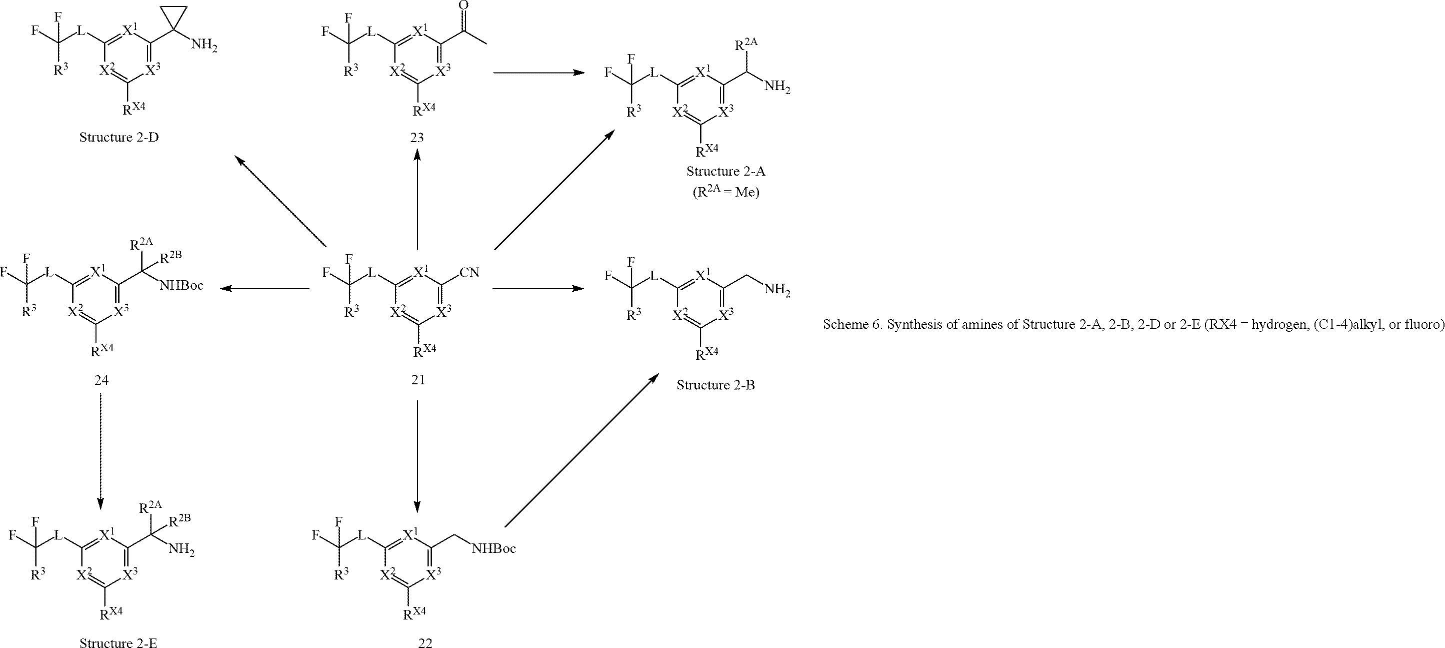

Aldehydes 8-A can be prepared as described in Scheme 7. Thus, alcohol 25 can be reacted with an alkylating agent like alkylsulfonate, alkylbromide, or alkyliodide in the presence of a base like Cs₂CO₃ or K₂CO₃ in a solvent like DMF to give an aldehyde 8-A. Similarly, an alcohol 26 can be converted into the corresponding bromide 18-A.

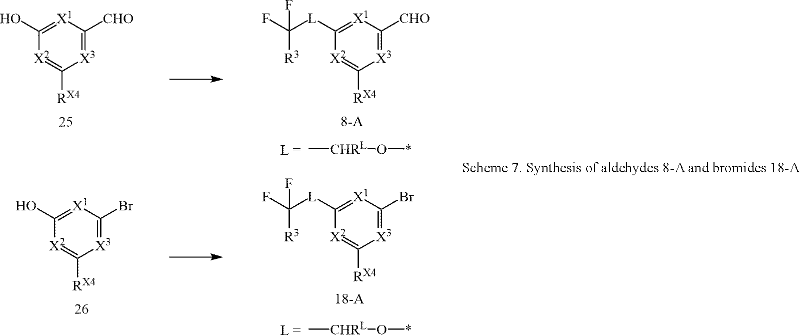

Nitriles 21-A are obtained through a S_{N}Ar reaction between a chloro or fluoro nitrile 27 and an alcohol like trifluoroethanol in the presence of a base like sodium hydride in a solvent like THF (Scheme 8). Alternatively, nitrile 27 can undergo a S_{N}Ar reaction with an amine (or the corresponding HCl salt) in a solvent like NMP and a base like NEt₃ under microwave irradiation to yield a nitrile 21-B. Finally, a cyanation between chloro or bromo derivative 28 and ZnCN₂ in the presence of a palladium catalyst like Pd₂(dba)₃ and a ligand like dppf in a solvent like DMF give a nitrile 21.

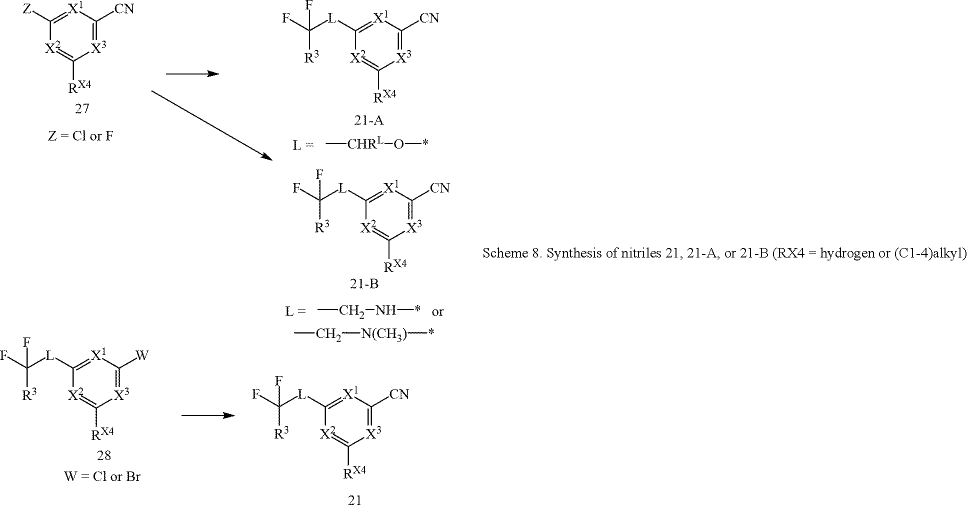

An amine of Structure 2 can also be prepared by methods described in Scheme 9. Thus, a Boc-protected amine 30 can be treated with an alkylating agent like alkyl bromide or alkyl iodide in the presence of a base or a silver salt like Ag₂O to give a Boc-protected amine 31. The Boc-protecting group is then cleaved under acidic conditions like TFA in DCM or 4 M HCl in dioxane to give an amine of Structure 2 (or the corresponding salt, like HCl or TFA salt). Alternatively, an aldehyde 8 can undergo a reductive amination with an amine 32 in a solvent like DCM and in the presence of a reducing agent like NaBH(OAc)₃ and a base like DIPEA to give an amine of Structure 2, wherein R^{2A} and R^{2B} represent hydrogen.

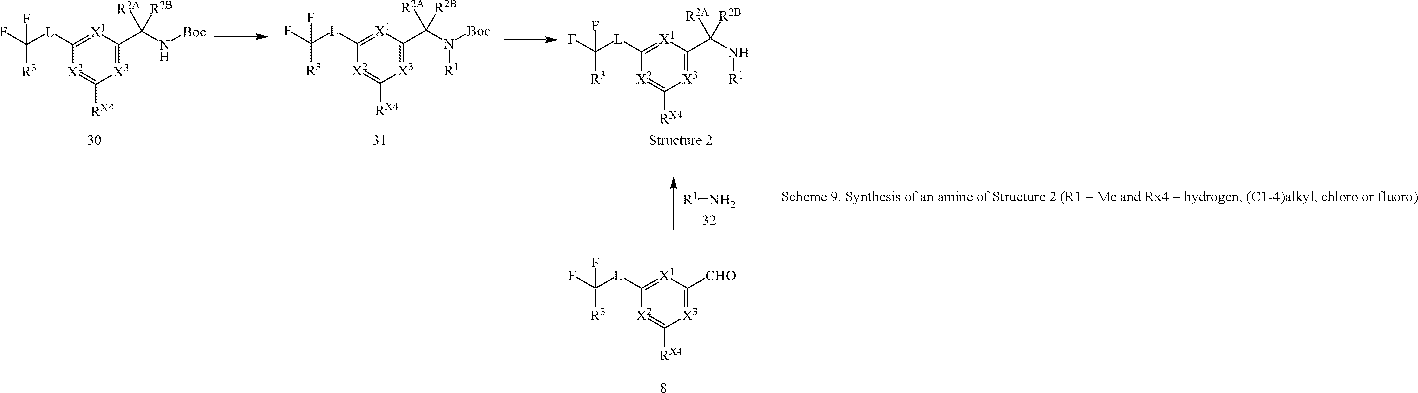

### Experimental Section

Abbrevations (as used herein and in the description above)

| | |
|---|---|
| anh. | anhydrous |
| Ac | acetyl |
| aq. | aqueous |
| Boc | tert.-butyloxycarbonyl |
| Bu | butyl |
| CDI | 1,1′-carbonyldiimidazole |
| comb. | Combined |
| d | day(s) |
| dba | dibenzylideneacetone |
| DCM | dichloromethane |
| DIPEA | N-ethyldiisopropylamine |
| DMA | dimethylacetamide |
| DMF | dimethylformamide |
| DMSO | dimethylsulfoxide |
| dppf | 1,1′-ferrocenediyl-bis(diphenylphosphine) |
| eq | equivalent |
| Et | ethyl |
| FBS | fetal bovine serum |
| FLIPR | Fluorescent imaging plate reader |
| Fluo-8-AM h | bis(acetoxymethyl) 2,2′-((4-(6-(acetoxymethoxy)-3-oxo-3H-xanthen-9-yl)-2-(2-(bis(2-acetoxymethoxy)-2-oxoethyl)amino)phenoxy)ethoxy)phenyl)azanediyl)diacetate hour(s) |
| HATU | 2-(7-Aza-1H-benzotriazole-1-yl)-1,1,3,3-tetramethyluronium hexafluorophosphate |
| HBSS | Hank’s balanced salt solution |
| HEK293 | Human embryonic kidney 293 cells |
| HEPES | 4-(2-hydroxyethyl)-piperazine-1-ethanesulfonic acid |
| Hept | heptane(s) |
| HV | High vacuum |
| HPLC | high performance liquid chromatography |
| iPr | isopropyl |
| lr(p-F(t-Bu)-ppy)₃ | tris (2- (3-tert-butylphenyl) -4-tert-butylpyridine) iridium |
| [lr{dF(CF₃)ppy}₂(dtbpy)]PF₆ | [4,4′-Bis(1,1-dimethylethyl)-2,2′-bipyridine-N1,N1′]bis[3,5-difluoro-2-[5-(trifluoromethyl)-2-pyridinyl-N] phenyl-C]lridium(III) hexafluorophosphate |
| LC | liquid chromatography |
| LED | light-emitting diode |
| M | molarity [mol L⁻¹] |
| Me | methyl |
| MS | mass spectroscopy |
| min | minute(s) |
| N | normality |
| NiCl₂•glyme | Nickel(II) chloride ethylene glycol dimethyl ether complex |
| NMDA | N-methyl-D-aspartate |
| NMP | N-methyl-2-pyrrolidone |
| NMR | Nuclear magnetic resonance |
| org. | organic |
| PBS | phosphate-buffered saline |
| PG | protecting group |
| Ph | phenyl |
| Prep. | Preparative |
| Ra/Ni | Raney-Nickel |
| rpm | rotations per minute |
| rt | room temperature |
| sat. | saturated |
| sec | second(s) |
| SFC | supercritical fluid chromatography |
| soln. | solution |
| TBME | tert-butyl methyl ether |
| tBu | tert-butyl |
| TFA | trifluoroacetic acid |
| THF | tetrahydrofuran |
| t_{R} | retention time |
| UPLC | Ultra performance liquid chromatography |
| UV | ultraviolet |
| XE-991 | 10,10-bis(4-pyridinylmethyl)-9(10H)-anthracenone |

### I. Chemistry

The following examples illustrate the preparation of biologically active compounds of the invention but do not at all limit the scope thereof.

General remarks: All solvents and reagents are used as obtained from commercial sources unless otherwise indicated. Temperatures are indicated in degrees Celsius (°C). Unless otherwise indicated, the reactions take place at room temperature (rt) under an argon or nitrogen atmosphere and are run in a flame dried round-bottomed flask equipped with a magnetic stir bar. In mixtures, relations of parts of solvent or eluent or reagent mixtures in liquid form are given as volume relations (v/v), unless indicated otherwise.

### Characterization Methods Used

LC-MS 1

LC-MS-conditions: Analytical. Pump: Waters Acquity Binary, Solvent Manager, MS: Waters SQ Detector or Xevo TQD, DAD: Acquity UPLC PDA Detector. Column: Acquity UPLC CSH C18 1.7 um, 2.1 × 50 mm from Waters, thermostated in the Acquity UPLC Column Manager at 60° C. Eluents: A1: H₂O + 0.05% formic acid; B1: MeCN + 0.045% formic acid. Method: Gradient: 2% B to 98% B over 2.0 min. Flow: 1.0 mL/min. Detection at 214 nm and MS, retention time t_{R} is given in min.

### LC-MS 2 to 4

UPLC/MS analyses are performed on Acquity UPLC setup. The column temperature is 40° C.

The LC retention times are obtained using the following elution conditions: LC-MS 2: Analytical UPLC on a Agilent Zorbax RRHD SB-Aq (2.1×50 mm, 1.8 um); detection at 210 nM and MS; Gradient of water/ 0.04% TFA (A) and MeCN (B). The eluent flow rate was 0.8 mL/min and the characteristics of the eluting mixture proportion in function of the time t from start of the elution are summarized in the table below (a linear gradient being used between two consecutive time points):

| t (min) | 0 | 1.2 | 1.9 | 2.1 |
|---|---|---|---|---|
| Solvent A (%) | 95 | 5 | 5 | 95 |
| Solvent B (%) | 5 | 95 | 95 | 5 |

LC-MS 3: Analytical UPLC on a Waters Xbridge (4.6×30 mm, 2.5 um); detection at 210 nM and MS; Gradient of water/ 0.04% TFA (A) and MeCN (B). The eluent flow rate was 4.5 mL/min and the characteristics of the eluting mixture proportion in function of the time t from start of the elution are summarized in the table below (a linear gradient being used between two consecutive time points):

| | | | | |
|---|---|---|---|---|
| t (min) | 0 | 1.00 | 1.45 | 1.55 |
| Solvent A (%) | 95 | 5 | 5 | 95 |
| Solvent B (%) | 5 | 95 | 95 | 5 |

LC-MS 4: Analytical UPLC on a Waters BEH C18 (2.1×50 mm, 2.5 um); detection at 210 nM and MS; Gradient of water/ 0.04% NH₃ [c(NH₃) = 13 mmol/l] (A) and MeCN (B). The eluent flow rate was 0.8 mL/min and the characteristics of the eluting mixture proportion in function of the time t from start of the elution are summarized in the table below (a linear gradient being used between two consecutive time points):

| | | | | |
|---|---|---|---|---|
| t (min) | 0 | 1.2 | 1.9 | 2.1 |
| Solvent A (%) | 95 | 5 | 5 | 95 |
| Solvent B (%) | 5 | 95 | 95 | 5 |

### Preparative LC-MS Methods Used

Preparative HPLC/MS purifications are performed on a Gilson HPLC system, equipped with a Gilson 215 autosampler, Gilson 333/334 pumps, Finnigan AQA MS detector system, and a Dionex UV detector, using a Waters Xbridge C18 or an Waters Atlantis column, with a linear gradient of water/formic acid 0.02% (A) and MeCN (B) (acidic conditions) or water/ammonia 0.02% (A) and MeCN (B) (basic conditions).

### Combiflash

Flash column chromatography was performed using a combiflash from Teledyne ISCO.

### Preparative Chiral SFC Methods Used

Preparative chiral SFC purifications are performed on a Sepiatec Prep SFC 360 system. Following parameters were used:
Preparative chiral SFC 1: A ChiralPak IB column (30×250 mm, 5 um) was used. The modifier was iPrOH (12%), run for 5 min and at a flow rate of 160 mL/min. The following system settings were used: backpressure 100 bar, temperature pumphead 5° C., temperature fraction module 20° C., and temperature column department 40° C.
Preparative chiral SFC 2: A ChiralPak IH column (30×250 mm, 5 um) was used. The modifier was EtOH (15%), run for 3.3 min and at a flow rate of 160 mL/min. The following system settings were used: backpressure 100 bar, temperature pumphead 5° C., temperature fraction module 20° C., and temperature column department 40° C.
Preparative chiral SFC 3: A Regis (R,R)Whelk-O1column (30×250 mm, 5 um) was used. The modifier was EtOH (15%), run for 3.0 min and at a flow rate of 160 mL/min. The following system settings were used: backpressure 100 bar, temperature pumphead 5° C., temperature fraction module 20° C., and temperature column department 40° C.
Preparative chiral SFC 4: A ChiralPak IB column (30×250 mm, 5 um) was used. The modifier was EtOH (10%), run for 5.5 min and at a flow rate of 160 mL/min. The following system settings were used: backpressure 100 bar, temperature pumphead 5° C., temperature fraction module 20° C., and temperature column department 40° C.
Preparative chiral SFC 5: A Regis (R,R)Whelk-O1column (30×250 mm, 5 um) was used. The modifier was MeOH (20%), run for 4.0 min and at a flow rate of 160 mL/min. The following system settings were used: backpressure 100 bar, temperature pumphead 5° C., temperature fraction module 20° C., and temperature column department 40° C.
Preparative chiral SFC 6: A ChiralPak AD-H column (30×250 mm, 5um) was used. The modifier was EtOH (10%), run for 3.0 min and at a flow rate of 160 mL/min. The following system settings were used: backpressure 100 bar, temperature pumphead 5° C., temperature fraction module 20° C., and temperature column department 40° C.

### NMR

¹H-NMR spectra were recorded at rt with a Brucker NMR 500 spectrometer 1H (500 MHz) equipped with a Bruker’s DCH cryoprobe. Chemical shifts are reported in ppm downfield from tetramethylsilane using residual solvent signals as internal reference. The multiplicity is described as singulet s, doublet d, triplet t, quadruplet q, hextet h, or multiplet m. Broad signals are indicated as br.

### Example 1: 1-(3,3-Difluoro-Cyclobutyl)-3-(3-Trifluoromethyl-Benzyl)-Urea

To a solution of 3-(trifluoromethyl)benzylamine (18 mg, 0.1 mmol, 1.0 eq) in MeCN (0.5 mL), DIPEA (19 µL, 0.11 mmol, 1.1 eq) and a solution of CDI (32 mg, 0.2 mmol, 2.0 eq) in MeCN (0.2 mL) were added in sequence. The mixture was stirred at 60° C. for 3 hours. A solution of 3,3-difluorocyclobutan-1-amine (21 mg, 0.2 mmol, 2.0 eq) in MeCN (0.5 mL) and H₂O (0.1 mL) was added. The mixture was further stirred at 60° C. overnight. The mixture was allowed to cool to rt and purified by prep. HPLC (column: Waters XBridge, 30×75 mm, 10 um, UV/MS, basic conditions). LC-MS (1): t_{R} = 0.99 min; [M+H]⁺: 309.2.

### Example 2: 1-Bicyclo[1.1.1]Pent-1-yl-3-[1-(3-Trifluoromethyl-Phenyl)-Ethyl]-Urea

To a solution of bicyclo[1.1.1]pentan-1-amine hydrochloride(12 mg, 0.1 mmol, 1 eq) in MeCN (0.5 mL), DIPEA (34 µL, 0.2 mmol, 2 eq) and CDI (16 mg, 0.1 mmol, 1 eq) were added in sequence. The mixture was stirred at 60° C. for 1 hour. A solution of 1-(3-trifluoromethylphenyl)ethylamine (19 mg, 0.1 mmol, 1 eq) in MeCN (0.4 mL) and H₂O (0.1 mL) was added. The reaction mixture was further stirred at 60° C. overnight. The mixture was allowed to cool to rt and purified by prep. HPLC (column: Waters XBridge, 30×75 mm, 10 um, UV/MS, basic conditions). LC-MS (1): t_{R} = 1.08 min; [M+H]⁺: 299.2.

Example 3 to Example 5 were synthesized using the appropriate amine or amine salt (HCI or TFA) derivative and following the procedure described in Example 2. LC-MS data of Example 3 to Example 5 are listed in the table below. The LC-MS conditions used were LC-MS (1).

| Example N° | Name | t_{R} | [M+H]⁺ |
|---|---|---|---|
| 3 | 1-(3-Difluoromethyl-cyclobutyl)-3-[1-(3-trifluoromethylphenyl)-ethyl]-urea | 1.05 | 337.3 |
| 4 | 1-(3-Fluoro-bicyclo[1.1.1]pent-1-yl)-3-[1-(3-trifluoromethylphenyl)-ethyl]-urea | 1.07 | 317.2 |
| 5 | 1-(3-Difluoromethyl-bicyclo[1.1.1]pent-1-yl)-3-[1-(3-trifluoromethyl-phenyl)-ethyl]-urea | 1.10 | 349.3 |

### Example 6: 1-Bicyclo[1.1.1]Pent-1-yl-3-[1-(3-Trifluoromethoxy-Phenyl)-Ethyl]-Urea

To a solution of bicyclo[1.1.1]pentan-1-amine hydrochloride (12 mg, 0.1 mmol, 1 eq) in MeCN (0.5 mL), DIPEA (34 µL, 0.2 mmol, 2 eq) and CDI (16 mg, 0.1 mmol, 1 eq) were added in sequence. The mixture was stirred at 60° C. for 1 hour. A solution of 1-(3-(trifluoromethoxy)phenyl)ethanamine (21 mg, 0.1 mmol, 1 eq) in MeCN (0.4 mL) and H₂O (0.1 mL) was added. The reaction mixture was further stirred at 60° C. overnight. The mixture was allowed to cool to rt and purified by prep. HPLC (column: Waters XBridge, 30×75 mm, 10 um, UV/MS, basic conditions). LC-MS (1): t_{R} = 1.11 min; [M+H]⁺: 315.2.

Example 7 to Example 8 were synthesized using the appropriate amine or amine salt (HCI or TFA) derivative and following the procedure described in Example 6. LC-MS data of Example 7 to Example 8 are listed in the table below. The LC-MS conditions used were LC-MS (1).

| Example N° | Name | t_{R} | [M+H]⁺ |
|---|---|---|---|
| 7 | 1-(3-Difluoromethyl-cyclobutyl)-3-[1-(3-trifluoromethoxyphenyl)-ethyl]-urea | 1.08 | 353.2 |
| 8 | 1-(3-Fluoro-bicyclo[1.1.1]pent-1-yl)-3-[1-(3-trifluoromethoxyphenyl)-ethyl]-urea | 1.10 | 333.2 |

### Example 9: 1-[2,2-Difluoro-1-(3-Trifluoromethyl-Phenyl)-Ethyl]-3-(3-Hydroxy-3-Trifluoromethyl-Cyclobutyl)-Urea

To a solution of 3-amino-1-(trifluoromethyl)cyclobutan-1-ol (19 mg, 0.12 mmol, 1.5 eq) in MeCN (0.1 mL), a solution of CDI (20 mg, 0.12 mmol, 1.5 eq) in MeCN (0.2 mL) was added. The reaction mixture was stirred at rt for 2 hours. A solution of 2,2-difluoro-1-[3-(trifluoromethyl)phenyl]ethan-1-amine (19 mg, 0.08 mmol, 1.0 eq) and DIPEA (15 µL, 0.09 mmol, 1.1 eq) in MeCN (0.5 mL) and H₂O (0.1 mL) was added. The mixture was stirred at rt overnight. The mixture was purified by prep. HPLC (column: Waters XBridge, 30×75 mm, 10 um, UV/MS, basic conditions). LC-MS (1): t_{R} = 1.01 min; [M+H]⁺: 407.3.

### Example 10: 1-(3,3-Difluoro-1-Methyl-Cyclobutyl)-3-[2,2-Difluoro-1-(3-TrifluoromethylPhenyl)-Ethyl]-Urea

The product was synthesized using 3-3-difluoro-1-methylcyclobutanamine-hydrochloride and following the procedure described in Example 9. LC-MS (1): t_{R} = 1.13 min; [M+H]⁺: 373.3.

### Example 11: 1-Bicyclo[1.1.1]Pent-1-yl-3-[1-(3-Difluoromethoxy-Phenyl)-Ethyl]-Urea

To a solution of 1-(3-(difluoromethoxy)phenyl)ethan-1-amine hydrochloride (37 mg, 0.1 mmol, 1 eq) in MeCN (0.5 mL), DIPEA (51 µL, 0.3 mmol, 3 eq) and CDI (32 mg, 0.2 mmol, 2 eq) were added in sequence. The reaction was stirred at rt for 1 h. A solution of bicyclo[1.1.1]pentan-1-amine hydrochloride in MeCN (0.4 mL) and H₂O (0.1 mL) was added. The reaction mixture was stirred at rt for 1 hour. The mixture was purified by prep. HPLC (column: Waters XBridge, 30×75 mm, 10 um, UV/MS, basic conditions). LC-MS (1): t_{R} = 1.01 min; [M+H]⁺: 297.3.

### Example 12: 1-(3-Difluoromethyl-Bicyclo[1.1.1]Pent-1-yl)-3-[2-Hydroxy-1-(3-Trifluoromethyl-Phenyl)-Ethyl]-Urea

To a solution of 3-(difluoromethyl)bicyclo[1.1.1]pentan-1-amine hydrochloride (25 mg, 0.15 mmol, 1.0 eq) in MeCN (0.8 mL), DIPEA (92 µL, 0.53 mmol, 3.5 eq) and CDI (37 mg, 0.23 mmol, 1.5 eq) were added in sequence. The mixture was stirred at 50° C. for 40 min. 2-Amino-2-(3-trifluoromethyl-phenyl)-ethanol (31 mg, 0.15 mmol, 1.0 eq) was added. The reaction mixture was stirred at 80° C. for 18 hours. The mixture was allowed to cool to rt and purified by prep. HPLC (column: Waters XBridge, 30×75 mm, 10 um, UV/MS, basic conditions). LC-MS (1): t_{R} = 0.96 min; [M+H]⁺: 365.2.

Example 13 to Example 16 were synthesized using the appropriate amine or amine salt (HCI or TFA) derivative and following the procedure described in Example 12. LC-MS data of Example 13 to Example 16 are listed in the table below. The LC-MS conditions used were LC-MS (1).

| Example N° | Name | t_{R} | [M+H]⁺ |
|---|---|---|---|
| 13 | 1-(3-Difluoromethyl-bicyclo[1.1.1]pent-1-yl)-3-[2,2-difluoro-1-(3-trifluoromethyl-phenyl)-ethyl]-urea | 1.12 | 385.3 |
| 14 | 1-(3-Difluoromethyl-bicyclo[1.1.1]pent-1-yl)-3-[2-methoxy-1-(3-trifluoromethyl-phenyl)-ethyl]-urea | 1.08 | 379.3 |
| 15 | 1-(3-Difluoromethyl-bicyclo[1.1.1]pent-1-yl)-3-(2-fluoro-3-trifluoromethyl-benzyl)-urea | 1.07 | 353.2 |
| 16 | 1-(3-Difluoromethyl-bicyclo[1.1.1]pent-1-yl)-3-(3-fluoro-5-trifluoromethyl-benzyl)-urea | 1.09 | 353.2 |

### Example 17: 1-(3-Fluoro-Bicyclo[1.1.1]Pent-1-yl)-3-[3-(2,2,2-Trifluoro-Ethoxy)-Benzyl]-Urea

A solution of (3-(2,2,2-trifluoroethoxy)phenyl)methanamine (14 mg, 0.07 mmol, 1.0 eq) in MeCN (0.4 mL) was treated at rt with DIPEA (43 µL, 0.25 mmol, 3.5 eq) followed by CDI (12 mg, 0.07 mmol, 1.05 eq) and the resulting mixture was stirred at 50° C. for 30 min. The resulting mixture was treated with 3-fluorobicyclo[1.1.1]pentan-1-amine hydrochloride (10 mg, 0.07 mmol, 1.0 eq) and the reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 1.02 min; [M+H]⁺: 333.3.

Example 18 to Example 19 were synthesized using the appropriate amine or amine salt (HCI or TFA) derivative and following the procedure described in Example 17. LC-MS data of Example 18 to Example 19 are listed in the table below. The LC-MS conditions used were LC-MS (1).

| Example N° | Name | t_{R} | [M+H]⁺ |
|---|---|---|---|
| 18 | 1-(3-Difluoromethyl-cyclobutyl)-3-[3-(2,2,2-trifluoro-ethoxy)-benzyl]-urea | 1.01 | 353.3 |
| 19 | 1-[3-(2,2,2-Trifluoro-ethoxy)-benzyl]-3-(3-trifluoromethyl-bicyclo[1.1.1]pent-1-yl)-urea | 1.14 | 383.3 |

### Example 20: 1-(3-Difluoromethoxy-Benzyl)-3-(3-Fluoro-Bicyclo[1.1.1]Pent-1-yl)-Urea

A solution of 3-(difluoromethoxy)benzylamine (12 mg, 0.07 mmol, 1.0 eq) in MeCN (0.4 mL) was treated at rt with DIPEA (43 µL, 0.25 mmol, 3.5 eq) followed by CDI (12 mg, 0.07 mmol, 1.05 eq) and the resulting mixture was stirred at 50° C. for 30 min. The resulting mixture was treated with 3-fluorobicyclo[1.1.1]pentan-1-amine hydrochloride (10 mg, 0.07 mmol, 1.0 eq) and the reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 0.95 min; [M+H]⁺: 301.2.

Example 21 to Example 22 were synthesized using the appropriate amine or amine salt (HCI or TFA) derivative and following the procedure described in Example 20. LC-MS data of Example 21 to Example 22 are listed in the table below. The LC-MS conditions used were LC-MS (1).

| Example N° | Name | t_{R} | [M+H]⁺ |
|---|---|---|---|
| 21 | 1-(3-Difluoromethoxy-benzyl)-3-(3-difluoromethyl-cyclobutyl)-urea | 0.94 | 321.2 |
| 22 | 1-(3-Difluoromethoxy-benzyl)-3-(3-trifluoromethyl-bicyclo[1.1.1]pent-1-yl)-urea | 1.08 | 351.2 |

### Example 23: 1-(3-Trifluoromethoxy-Benzyl)-3-(3-Trifluoromethyl-Bicyclo[1.1.1]Pent-1-yl)-Urea

A solution of 3-(trifluoromethoxy)benzylamine (22 mg, 0.07 mmol, 1.0 eq) in MeCN (0.4 mL) was treated at rt with DIPEA (43 µL, 0.25 mmol, 3.5 eq) followed by CDI (12 mg, 0.07 mmol, 1.05 eq) and the resulting mixture was stirred at 50° C. for 30 min. The resulting mixture was treated with 3-(trifluoromethyl)bicyclo[1.1.1]pentan-1-amine hydrochloride (14 mg, 0.07 mmol, 1.0 eq) and the reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 1.17 min; [M+H]⁺: 369.2.

Example 24 to Example 26 were synthesized using the appropriate amine or amine salt (HCI or TFA) derivative and following the procedure described in Example 23. LC-MS data of Example 24 to Example 26 are listed in the table below. The LC-MS conditions used were LC-MS (1).

| Example N° | Name | t_{R} | [M+H]⁺ |
|---|---|---|---|
| 24 | 1-(3-Fluoro-bicyclo[1.1.1]pent-1-yl)-3-(3-trifluoromethoxybenzyl)-urea | 1.05 | 319.2 |
| 25 | 1-(3-Difluoromethyl-cyclobutyl)-3-(3-trifluoromethoxy-benzyl)-urea | 1.03 | 339.2 |
| 26 | 1-Bicyclo[1.1.1]pent-1-yl-3-(3-trifluoromethoxy-benzyl)-urea | 1.06 | 301.2 |

### Example 27: 1-(3-Difluoromethyl-Benzyl)-3-(3-Difluoromethyl-Cyclobutyl)-Urea

To a solution of [3-(difluoromethyl)phenyl]methanamine hydrochloride (14 mg, 0.07 mmol, 1.0 eq) in MeCN (0.4 mL), DIPEA (43 µL, 0.25 mmol, 3.5 eq) and CDI (17 mg, 0.11 mmol, 1.5 eq) were added in sequence. The resulting mixture was stirred at 50° C. for 1.5 h. 3-(difluoromethyl)cyclobutan-1-amine hydrochloride (11 mg, 0.07 mmol, 1.0 eq) was added. The reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 0.92 min; [M+H]⁺: 305.2.

### Example 28: 1-(3-Difluoromethyl-Benzyl)-3-(3-Trifluoromethyl-Bicyclo[1.1.1]Pent-1-yl)-urea

The product was synthesized using 3-(trifluoromethyl)bicyclo[1.1.1]pentan-1-amine hydrochloride and following the procedure described in Example 27. LC-MS (1): t_{R} = 1.06 min; [M+H]⁺: 335.2.

### Example 29: 1-(3-Difluoromethyl-Cyclobutyl)-3-(2-Trifluoromethoxy-Pyridin-4-Ylmethyl)-Urea

To a solution of (2-(trifluoromethoxy)pyridin-4-yl)methanamine hydrochloride (23 mg, 0.07 mmol, 1.0 eq) in MeCN (0.4 mL), DIPEA (43 µL, 0.25 mmol, 3.5 eq) and CDI (12 mg, 0.07 mmol, 1.05 eq) were added in sequence. The resulting mixture was stirred at 50° C. for 1.5 h. 3-(difluoromethyl)cyclobutan-1-amine hydrochloride (11 mg, 0.07 mmol, 1.0 eq) was added. The reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 0.91 min; [M+H]⁺: 340.2.

### Example 30: 1-(2-Trifluoromethoxy-Pyridin-4-Ylmethyl)-3-(3-Trifluoromethyl-Bicyclo[1.1.1]Pent-1-yl)-Urea

The product was synthesized using 3-(trifluoromethyl)bicyclo[1.1.1]pentan-1-amine hydrochloride and following the procedure described in Example 29. LC-MS (1): t_{R} = 1.06 min; [M+H]⁺: 370.2.

### Example 31: (±)-1-(3-Difluoromethyl-Cyclobutyl)-3-{2-Methoxy-1-[2-(2,2,2-Trifluoro-Ethoxy)-Pyridin-4-yl]-Ethyl}-Urea

To a solution of (±)-2-methoxy-1-(2-(2,2,2-trifluoroethoxy)pyridin-4-yl)ethan-1-amine hydrochloride (30 mg, 0.11 mmol, 1.0 eq) in MeCN (0.6 mL), DIPEA (64 µL, 0.37 mmol, 3.5 eq) and CDI (18 mg, 0.11 mmol, 1.05 eq) were added in sequence. The resulting mixture was stirred at 50° C. for 3 h. 3-(difluoromethyl)cyclobutan-1-amine hydrochloride (17 mg, 0.11 mmol, 1.0 eq) was added. The reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 1.00 min; [M+H]⁺: 398.3.

### Example 32: (±)-1-{2-Methoxy-1-[2-(2,2,2-Trifluoro-Ethoxy)-Pyridin-4-yl]-Ethyl}-3-(3-Trifluoromethyl-Cyclobutyl)-Urea

The product was synthesized using 3-(trifluoromethyl)cyclobutan-1-amine hydrochloride and following the procedure described in Example 31. LC-MS (1): t_{R} = 1.07 min; [M+H]⁺: 416.3.

### Example 33: (±)-1-[1-(2-Difluoromethoxy-Pyridin-4-yl)-Ethyl]-3-(3-Difluoromethyl-Cyclobutyl)-Urea

To a solution of (±)-1-(2-(difluoromethoxy)pyridin-4-yl)ethan-1-amine (22 mg, 0.07 mmol, 1.0 eq) in MeCN (0.4 mL), DIPEA (43 mL, 0.245 mmol, 3.5 eq) and CDI (12 mg, 0.07 mmol, 1.05 eq) were added in sequence. The resulting mixture was stirred at 50° C. for 3 h. 3-(difluoromethyl)cyclobutan-1-amine hydrochloride (11 mg, 0.07 mmol, 1.0 eq) was added. The reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 0.93 min; [M+H]⁺: 336.3.

### Example 34: (±)-1-{1-[2-Methyl-6-(2,2,2-Trifluoro-Ethoxy)-Pyrimidin-4-yl]-Ethyl}-3-(3-Trifluoromethyl-Bicyclo[1.1.1]Pent-1-yl)-Urea

To a solution 3-(trifluoromethyl)bicyclo[1.1.1]pentan-1-amine hydrochloride (126 mg, 0.64 mmol, 1.2 eq) in MeCN (2 mL), DIPEA (0.186 mL, 1.06 mmol, 2.0 eq) and CDI (103 mg, 0.638 mmol, 1.2 eq) were added in sequence. The mixture was stirred at 50° C. for 1 h. A solution of (±)-1-(2-methyl-6-(2,2,2-trifluoroethoxy)pyrimidin-4-yl)ethan-1-amine (125 mg, 0.531 mmol, 1.0 eq) in MeCN (1.1 mL) was added. The reaction mixture was stirred at 80° C. overnight. The mixture was allowed to cool to rt and purified by prep. HPLC (column: Waters XBridge, 30×75 mm, 10 um, UV/MS, basic conditions). LC-MS (1): t_{R} = 1.12 min; [M+H]⁺: 413.3.

### Example 35: 1-Bicyclo[1.1.1]Pent-1-yl-3-(3-Trifluoromethyl-Benzyl)-Urea

To a solution of bicyclo[1.1.1]pentan-1-amine hydrochloride (10 mg, 0.08 mmol, 1 eq) in THF (1 mL), NEt₃ (45 µL, 0.32 mmol, 4 eq) and 4-nitrophenyl (3-(trifluoromethyl)benzyl)carbamate (27 mg, 0.08 mmol, 1 eq) were added in sequence. The resulting mixture was stirred at rt overnight. The mixture was concentrated in vacuo. The residue was purified by prep. HPLC (column: Waters XBridge, 30×75 mm, 10 um, UV/MS, basic conditions). LC-MS (1): t_{R} = 1.03 min; [M+H]⁺: 285.2.

Example 36 to Example 42 were synthesized using the appropriate amine or amine salt (HCI or TFA) derivative and following the procedure described in Example 35. LC-MS data of Example 36 to Example 42 are listed in the table below. The LC-MS conditions used were LC-MS (1).

| Example N° | Name | t_{R} | [M+H]⁺ |
|---|---|---|---|
| 36 | 1-(3-Fluoro-bicyclo[1.1.1]pent-1-yl)-3-(3-trifluoromethyl-benzyl)-urea | 1.02 | 303.2 |
| 37 | 1-(3-Trifluoromethyl-benzyl)-3-(3-trifluoromethyl-bicyclo[1.1.1]pent-1-yl)-urea | 1.14 | 353.2 |
| 38 | 1-(3-Difluoromethyl-cyclobutyl)-3-(3-trifluoromethyl-benzyl)-urea | 1.00 | 323.2 |
| 39 | 1-(3-Methyl-bicyclo[1.1.1]pent-1-yl)-3-(3-trifluoromethyl-benzyl)-urea | 1.10 | 299.2 |
| 40 | 1-(3-Fluoromethyl-bicyclo[1.1.1]pent-1-yl)-3-(3-trifluoromethyl-benzyl)-urea | 1.02 | 317.2 |
| 41 | 1-(3-Trifluoromethyl-benzyl)-3-(3-trifluoromethyl-cyclobutyl)-urea | 1.07 | 341.2 |
| 42 | 1-(3-Hydroxy-3-trifluoromethyl-cyclobutyl)-3-(3-trifluoromethyl-benzyl)-urea | 0.94 | 357.2 |

### Example 43: 1-Bicyclo[1.1.1]Pent-1-yl-3-[2-(2,2,2-Trifluoro-Ethoxy)-Pyridin-4-Ylmethyl]-Urea

To a solution of bicyclo[1.1.1]pentan-1-amine hydrochloride (30 mg, 0.08 mmol, 1 eq) in THF (1 mL), NEtₛ (45 µL, 0.32 mmol, 4 eq) and 4-nitrophenyl ((2-(2,2,2-trifluoroethoxy)pyridin-4-yl)methyl)carbamate (30 mg, 0.08 mmol, 1 eq) were added in sequence. The resulting mixture was stirred at rt overnight. The mixture was concentrated in vacuo. The residue was purified by prep. HPLC (column: Waters XBridge, 30×75 mm, 10 um, UV/MS, basic conditions). LC-MS (1): t_{R} = 0.98 min; [M+H]⁺: 316.2.

Example 44 to Example 46 were synthesized using the appropriate amine or amine salt (HCI or TFA) derivative and following the procedure described in Example 43. LC-MS data of Example 44 to Example 46 are listed in the table below. The LC-MS conditions used were LC-MS (1).

| Example N° | Name | t_{R} | [M+H]⁺ |
|---|---|---|---|
| 44 | 1-(3-Fluoro-bicyclo[1.1.1]pent-1-yl)-3-[2-(2,2,2-trifluoro-ethoxy)-pyridin-4-ylmethyl]-urea | 0.97 | 334.2 |
| 45 | 1-[2-(2,2,2-Trifluoro-ethoxy)-pyridin-4-ylmethyl]-3-(3-trifluoromethyl-bicyclo[1.1.1]pent-1-yl)-urea | 1.10 | 384.2 |
| 46 | 1-(3-Difluoromethyl-cyclobutyl)-3-[2-(2,2,2-trifluoro-ethoxy)-pyridin-4-ylmethyl]-urea | 0.96 | 354.2 |

### Example 47: 1-(3-Difluoromethyl-Bicyclo[1.1.1]Pent-1-yl)-3-(3-Trifluoromethyl-Benzyl)-Urea

To an ice-cooled solution of 3-(difluoromethyl)bicyclo[1.1.1]pentan-1-amine hydrochloride (125 mg, 0.74 mmol, 1 eq) in DCM (20 mL), NEtₛ (0.31 mL, 2.21 mmol, 3 eq) and 1-(isocyanatomethyl)-3-(trifluoromethyl)benzene (156 mg, 0.74 mmol, 1 eq) were added dropwise in sequence. The resulting mixture was stirred at 0° C. for 2 hours. The mixture was diluted with sat. aq. NaHCO₃ soln. and extracted with DCM (3x). The comb. org. layers were washed with sat. aq. NaCl soln., dried over MgSO₄, filtered and concentrated in vacuo. The residue was purified by prep. HPLC (column : Waters XBridge, 30×50 mm, 10 um, UV/MS, basic conditions). LC-MS (1): t_{R} = 1.06 min; [M+H]⁺: 335.2.

### Example 48: 1-Bicyclo[2.1.1]Hex-1-yl-3-(3-Trifluoromethyl-Benzyl)-Urea

To a solution of bicyclo[2.1.1]hexan-1-amine hydrochloride (27 mg, 0.2 mmol, 1.0 eq) in MeCN (0.7 mL), DIPEA (87 µL, 0.5 mmol, 2.5 eq) and 1-(isocyanatomethyl)-3-(trifluoromethyl)benzene (64 mg, 0.3 mmol, 1.5 eq) were added in sequence. The reaction mixture was stirred at rt overnight. The mixture was purified by prep. HPLC (column : Waters XBridge, 30×50 mm, 10 um, UV/MS, basic conditions). LC-MS (1): t_{R} = 1.09 min; [M+H]⁺: 299.2.

### Example 49: 1-(3,3-Difluoro-1-Methyl-Cyclobutyl)-3-(3-Trifluoromethyl-Benzyl)-Urea

The product was synthesized using 3,3-difluoro-1-methylcyclobutanamine hydrochloride and following the procedure described in Example 48. LC-MS (1): t_{R} = 1.06 min; [M+H]⁺: 323.2.

### Example 50: 1-(3-(Trifluoromethyl)Benzyl)-3-(3-(Trifluoromethyl)Cyclobutyl)Urea (Stereoisomer 1) and Example 51: 1-(3-(Trifluoromethyl)Benzyl)-3-(3-(Trifluoromethyl)Cyclobutyl)Urea (Stereoisomer 2)

1-Trifluoromethyl-benzyl)-3-trifluoromethyl-cyclobutyl)-urea was separated by the preparative chiral SFC 1 method to give Example 50 (first eluting, t_{R} = 2.9 min) and Example 51 (second eluting, t_{R} = 4.0 min). Example 50 (LC-MS (1): t_{R} = 1.07 min; [M+H]⁺: 341.2), ¹H-NMR (500 MHz, DMSO) δ: 7.54-7.59 (m, 4 H), 6.51-6.53 (m, 2 H), 4.27 (d, J = 6.1 Hz, 2 H), 4.17-4.23 (m, 1 H), 2.85-3.12 (m, 1 H), 2.33-2.38 (m, 2 H), 2.18-2.24 (m, 2 H). Example 51 (LC-MS (1): t_{R} = 1.07 min; [M+H]⁺: 341.2), ¹H-NMR (500 MHz, DMSO) δ: 7.53-7.59 (m, 4 H), 6.48-6.65 (m, 1 H), 6.39-6.48 (m, 1 H), 4.28 (d, J = 6.1 Hz, 2 H), 3.92-4.19 (m, 1 H), 2.76-2.88 (m, 1 H), 2.35-2.42 (m, 2 H), 1.88-1.94 (m, 2 H).

### Example 52: 1-(3-(Difluoromethyl)Cyclobutyl)-3-(3-(Trifluoromethyl)Benzyl)Urea (Stereoisomer 1) and Example 53: 1-(3-(Difluoromethyl)Cyclobutyl)-3-(3-(Trifluoromethyl)Benzyl)Urea (Stereoisomer 2)

1-Difluoromethyl-cyclobutyl)-3-trifluoromethyl-benzyl)-urea was separated by the preparative chiral SFC 2 method to give Example 52 (first eluting, t_{R} = 2.1 min) and Example 53 (second eluting, t_{R} = 2.6 min). Example 52 (LC-MS (1): t_{R} = 1.01 min; [M+H]⁺: 323.2) );¹ H-NMR (500 mHz, DMSO) δ: 7.54-7.59 (m, 4 H), 6.38-6.43 (m, 2 H), 5.99 (td, J = 57.3, 4.2 Hz, 1 H), 4.28 (d, J = 6.1 Hz, 2 H), 4.05 (h, J = 8.4 Hz, 1 H), 2.31-2.43 (m, 1 H), 2.23-2.28 (m, 2 H), 1.76-1.83 (m, 2 H). Example 53 (LC-MS (1): t_{R} = 1.01 min; [M+H]⁺: 323.2); ¹H-NMR (500 MHz, DMSO) δ: 7.54-7.59 (m, 4 H), 6.44-6.47 (m, 2 H), 6.18 (td, J = 57.2, 4.8 Hz, 1 H), 4.27 (d, J = 6.1 Hz, 2 H), 4.18 (h, J = 7.9 Hz, 1 H), 2.47-2.57 (m, 1 H), 2.21-2.26 (m, 2 H), 2.01-2.07 (m, 2 H).

### Example 54: 1-{(S)-1-[2-Methyl-6-(2,2,2-Trifluoro-Ethoxy)-Pyrimidin-4-yl]-Ethyl}-3-(3-Trifluoromethyl-Bicyclo[1.1.1]Pent-1-yl)-Urea

Racemic 1-{1-[2-Methyl-6-(2,2,2-trifluoro-ethoxy)-pyrimidin-4-yl]-ethyl}-3-(3-trifluoromethyl-bicyclo[1.1.1]pent-1-yl)-urea was separated by the preparative chiral SFC 3 method to give Example 54 (first eluting, t_{R} = 1.6 min) and 1-{(R)-1-[2-Methyl-6-(2,2,2-trifluoro-ethoxy)-pyrimidin-4-yl]-ethyl}-3-(3-trifluoromethyl-bicyclo[1.1.1]pent-1-yl)-urea (second eluting, t_{R} = 2.2 min). Example 54 (LC-MS (1): t_{R} = 1.12 min; [M+H]⁺: 413.3). The stereochemistry at the benzylic position has been assigned in analogy to Example 190 of PCT/EP2021/060918, meaning that the more active isomer was assumed to have (S)-configuration.

### Example 55: 1-(3-(Difluoromethyl)Cyclobutyl)-3-((2-(2,2,2-Trifluoroethoxy)Pyridin-4-yl)Methyl)Urea (Stereoisomer 1) and Example 56: 1-(3-(Difluoromethyl)Cyclobutyl)-3-((2-(2,2,2-Trifluoroethoxy)Pyridin-4-yl)Methyl)Urea (Stereoisomer 2)

1-Difluoromethyl-cyclobutyl)-3-[2-(2,2,2-trifluoro-ethoxy)-pyridin-4-ylmethyl]-urea was separated by the preparative chiral SFC 4 method to give Example 55 (first eluting, t_{R} = 3.6 min) and Example 56 (second eluting, t_{R} = 4.6 min). Example 55 (LC-MS (1): t_{R} = 0.96 min; [M+H]⁺: 354.3), ¹H-NMR (500 MHz, DMSO) δ: 8.10-8.11 (m, 1 H), 6.96 (dd, J = 5.3, 1.2 Hz, 1 H), 6.76 (br s, 1 H), 6.39-6.45 (m, 2 H), 6.00 (td, J = 57.3, 4.2 Hz, 1 H), 4.98 (q, J = 9.1 Hz, 2 H), 4.20 (d, J = 6.1 Hz, 2 H), 4.05 (h, J = 8.5 Hz, 1 H), 2.32-2.45 (m, 1 H), 2.17-2.32 (m, 2 H), 1.77-1.83 (m, 2 H). Example 56 (LC-MS (1): t_{R} = 0.96 min; [M+H]⁺: 354.2), ¹H-NMR (500 MHz, DMSO) δ: 8.10-8.11 (m, 1 H), 6.97 (dd, J = 5.2, 1.2 Hz, 1 H), 6.76 (br s, 1 H), 6.43-6.51 (m, 2 H), 6.18 (td, J = 57.2, 4.8 Hz, 1 H), 4.98 (q, J = 9.1 Hz, 2 H), 4.15-4.21 (m, 3 H), 2.53-2.59 (m, 1 H), 2.22-2.27 (m, 2 H), 1.92-2.13 (m, 2 H).

### Example 57: 1-(3-(Difluoromethyl)Cyclobutyl)-3-((S)-1-(3-(Trifluoromethoxy)Phenyl)Ethyl)Urea (Stereoisomer 1) and Example 58: 1-(3-(Difluoromethyl)Cyclobutyl)-3-((S)-1-(3-(Trifluoromethoxy)Phenyl)Ethyl)Urea (Stereoisomer 2)

1-Difluoromethyl-cyclobutyl)-3-[1-trifluoromethoxy-phenyl)-ethyl]-urea was first separated by preparative chiral SFC 4 method to give Fraction 1 (first eluting, t_{R} = 1.8 min) and Fraction 2 (second eluting, t_{R} = 2.9 min). Fraction 1 was further separated by preparative chiral SFC 1 method to give Example 57 (first eluting, t_{R} = 2.8 min) and Example 58 (second eluting, t_{R} = 3.8 min). Example 57 (LC-MS (1): t_{R} = 1.08 min; [M+H]⁺: 353.2), ¹H-NMR (500 MHz, DMSO) δ: 7.45 (t, J = 7.8 Hz, 1 H), 7.31 (d, J = 7.8 Hz, 1 H), 7.19-7.24 (m, 2 H), 6.35 (d, J = 8.0 Hz, 1 H), 6.16 (d, J = 8.3 Hz, 1 H), 5.99 (td, J = 57.3, 4.2 Hz, 1 H), 4.76 (quint, J = 7.1 Hz, 1 H), 3.98-4.03 (m, 1 H), 2.32-2.42 (m, 1 H), 2.20-2.26 (m, 2 H), 1.72-1.79 (m, 2 H), 1.31 (d, J = 7.1 Hz, 3 H). Example 58 (LC-MS (1): t_{R} = 1.08 min; [M+H]⁺: 353.3), ¹H-NMR (500 MHz, DMSO) δ: 7.45 (t, J = 7.9 Hz, 1 H), 7.32 (d, J = 7.9 Hz, 1 H), 7.19-7.24 (m, 2 H), 6.39 (d, J = 8.0 Hz, 1 H), 6.23 (d, J = 8.0 Hz, 1 H), 6.17 (td, J = 57.2, 4.8 Hz, 1 H), 4.74-4.79 (m, 1 H), 4.10-4.16 (m, 1 H), 2.48-2.53 (m, 1 H), 2.17-2.24 (m, 2 H), 1.97-2.04 (m, 2 H), 1.31 (d, J = 7.0 Hz, 3 H). Fraction 2 was further separated by preparative chiral SFC 6 method to give 1-(3-(difluoromethyl)cyclobutyl)-3-((R)-1-(3-(trifluoromethoxy)phenyl)ethyl)urea (first eluting stereoisomer, t_{R} = 1.7 min) and 1-(3-(difluoromethyl)cyclobutyl)-3-((R)-1-(3-(trifluoromethoxy)phenyl)ethyl)urea (second eluting stereoisomer, t_{R} = 2.3 min). The stereochemistry at the benzylic position has been assigned in analogy to Example 190 of PCT/EP2021/060918, meaning that the more active isomer was assumed to have (S)-configuration.

### Synthesis of 4-nitrophenyl (3-(trifluoromethyl)benzyl)carbamate

To an ice-cooled solution of 3-(trifluoromethyl)benzylamine (1.50 g, 8.4 mmol, 1 eq) and DIPEA (4.31 mL, 25.2 mmol, 3 eq) in THF (43 mL), 4-nitrophenyl chloroformate (1.74 g, 8.4 mmol, 1 eq) was added. The resulting mixture was stirred at 0° C. for 1 hour. The reaction mixture was diluted with water (25 mL) and EtOAc (25 mL). The layers were separated. The aq. phase was extracted with EtOAc (2 × 25 mL). The comb. org. phases were dried over MgSO₄ and concentrated in vacuo. The residue was purified by CombiFlash (column: 40 g, flow: 37 mL/min, Heptane 100% to Heptane + 20% EtOAc) to afford a pale yellow solid which was further triturated in heptane/EtOAc 8:2 to yield a white solid. LC-MS (2): t_{R} = 1.00 min; no ionization.

The following carbamate was synthesized using the appropriate amine and following the procedure described for 4-nitrophenyl (3-(trifluoromethyl)benzyl)carbamate. LC-MS data are listed in the table below. The LC-MS conditions used were LC-MS (2).

| Name | t_{R} | [M+H]⁺ |
|---|---|---|
| 4-nitrophenyl ((2-(2,2,2-trifluoroethoxy)pyridin-4-yl)methyl)carbamate | 0.97 | 372.15 |

### Synthesis of (3-(2,2,2-Trifluoroethoxy)Phenyl)Methanamine

### Step 1: 3-(2,2,2-Trifluoroethoxy)Benzaldehyde

To a solution of 3-hydroxybenzaldehyde (3.0 g, 24.6 mmol, 1.0 eq) and Cs₂CO₃ (12.0 g, 36.8 mmol, 1.5 eq) in DMF (45 mL), trifluoromethansulfonic acid 2.2,2-trifluoroethylester (4.25 ml, 29.5 mmol, 1.2 eq) was added dropwise. The reaction was stirred at rt for 2 h. The reaction was quenched with water and the mixture was extracted with Et;O. The comb. org. phases were dried over MgSO₄ and concentrated in vacuo to yield an orange oil. The product was used without further purification. LC-MS (3): t_{R} = 0.75 min; no ionization.

### Step 2: (:I:, E)-2-Methyl-N-(3-(2, 2, 2-Trifluoroethoxy)Benzylidene) Propane-2-Sulfinamide

A mixture of 3-(2,2,2-trifluoroethoxy)benzaldehyde (5.01 g, 24.5 mmol, 1 eq), (±)-2-methyl-2-propanesulfinamide (2.98 g, 24.5 mmol, 1 eq), and Ti(OEt)₄ (10.3 mL, 49.1 mmol, 2 eq) in THF (42 mL) was stirred at rt for 3 d. The reaction was quenched with sat. aq. NaCl soln. The resulting suspension was filtered and the solids rinsed with EtOAc. The filtrate was washed with sat. aq. NaCl soln., dried over MgSO₄, and concentrated in vacuo. The residue was purified by flash column chromatography (SiO₂, DCM) to give an orange solid. LC-MS (3): t_{R} = 0.87 min; [M+H]⁺: 307.98.

### Step 3: (±)-2-Methyl-N-(3-(2,2,2-Trifluoroethoxy)Benzyl)Propane-2-Sulfinamide

To a solution of (±,E)-2-methyl-N-(3-(2,2,2-trifluoroethoxy)benzylidene)propane-2-sulfinamide (6.85 g, 22.3 mmol, 1 eq) in MeOH (78 mL) and DCM (162 mL), NaBH₄ (5.06 g, 134 mmol, 6 eq) was added. The reaction mixture was stirred at rt for 10 min. The reaction was quenched with water. The mixture was extracted with DCM. The comb. org. phases were dried over MgSO₄ and concentrated in vacuo to afford a colorless oil. The product was used without further purification. LC-MS (3): t_{R} = 0.75 min; [M+H]⁺: 310.00.

### Step 4: (3-(2,2,2-Trifluoroethoxy)Phenyl)Methanamine

To an ice-cooled solution of (±)-2-methyl-N-(3-(2,2,2-trifluoroethoxy)benzyl)propane-2-sulfinamide (6.65 g, 21.5 mmol, 1 eq) in anhydrous methanol (80 mL), 4N HCI in dioxane (10.8 mL, 43 mmol, 2 eq) was added dropwise. The reaction mixture was stirred at 0° C. for 10 min and further at rt overnight. The yellow homogeneous reaction mixture was carefully concentrated to dryness under reduced pressure. The residue was partitioned between DCM (150 mL) and water (30 mL). Solid Na₂CO₃ (11.39 g, 107 mmol, 5 eq) was added. The layers were separated and the aq. phase was extracted with DCM (50 mL). The comb. org. phases were dried over MgSO₄ and concentrated in vacuo. The product was used without further purification. LC-MS (3): t_{R} = 0.46 min; [M+H]⁺: 206.06.

### Synthesis of 1-(3-(Difluoromethoxy)Phenyl)Ethan-1-Amine Hydrochloride

To a solution of 3-(difluoromethoxy)benzonitrile (1.0 g, 5.79 mmol, 1 eq) in THF (5 mL), 3.4 M methylmagnesium bromide in 2-methyltetrahydrofuran (5.11 mL, 17.4 mmol, 3 eq) was added dropwise. The mixture was stirred at rt for 2 hours. The reaction was cooled to 15° C. and quenched with MeOH (20 mL). NaBH₄ (438 mg, 11.6 mmol, 2 eq) was added and the mixture was stirred at rt overnight. 2 M aq. HCI soln. (30 mL) was added and the mixture was stirred at rt for 5 min. The organic solvents were removed in vacuo. The resulting solution was partitioned between DCM (50 mL) and sat. aq. NaHCO₃ soln. (30 mL). The layers were separated. The org. phase was treated with 1.25 M HCI in MeOH (20 mL) and concentrated in vacuo to give an oil. The product was used without further purification. LC-MS (4): t_{R} = 0.73 min; [M+H]⁺: 188.34.

### Synthesis of (2-(Trifluoromethoxy)Pyridin-4-yl)Methanamine Hydrochloride

### Step 1: (±,E)-2-Methyl-N-((2-(Trifluoromethoxy)Pyridin-4-yl)Methylene)Propane-2-Sulfinamide

To a mixture of 2-(trifluoromethoxy)pyridine-4-carbaldehyde (573 mg, 3 mmol, 1.0 eq) and (±)-2-methylpropane-2-sulfinamide (498 mg, 3.9 mmol, 1.3 eq) in THF (15 mL), titanium ethoxide (0.68 mL, 3.3 mmol, 1.1 eq) was added dropwise. The solution was stirred at rt for 17 hours. The yellow solution was diluted with water (20 mL) and DCM (10 mL). The resulting mixture was filtered. The layers were separated and the aq. phase was extracted with DCM (2 × 20 mL). The comb. org. phases were washed with H₂O (1 × 20 mL), sat. aq. NaCl soln. (1 × 20 mL), dried over MgSO₄, and concentrated in vacuo. The residue was purified by Combiflash (column: 40 g, flow: 40 mL/min, heptane to heptane/EtOAc 100:30) to give a white solid. LC-MS (2): t_{R} = 0.96 min; [M+H]⁺: 295.18.

### Step 2: (±)-2-Methyl-N-((2-(Trifluoromethoxy)Pyridin-4-yl)Methyl)Propane-2-Sulfinamide

To an ice-cooled solution of (±,E)-2-methyl-N-((2-(trifluoromethoxy)pyridin-4-yl)methylene)propane-2-sulfinamide (285 mg, 0.97 mmol, 1.0 eq) in MeOH (20 mL), sodium borohydride (55 mg, 1.45 mmol, 1.5 eq) was added. The mixture was stirred at 0° C. for 2.5 hours. The reaction mixture was concentrated in vacuo. The residue was partitioned between water (25 mL) and DCM (25 mL). The layers were separated. The aq. phase was extracted with DCM (2 × 25 mL). The comb. org. phases were dried over MgSO₄ and concentrated in vacuo. The product was used without further purification. LC-MS (2): t_{R} = 0.82 min; [M+H]⁺: 297.22.

### Step 3: (2-(Trifluoromethoxy)Pyridin-4-yl)Methanamine Hydrochloride

To an ice-cooled solution of (±)-2-methyl-N-((2-(trifluoromethoxy)pyridin-4-yl)methyl)propane-2-sulfinamide (279 mg, 0.94 mmol, 1 eq) in MeOH (20 mL), 4N HCI in dioxane (1.2 mL, 4.71 mmol, 5 eq) was added. The resulting mixture was stirred at 0° C. for 1 hour. The reaction mixture was concentrated in vacuo. The product was used crude for the next step. LC-MS (2): t_{R} = 0.40 min; [M+H]⁺: 193.28.

### Synthesis of (±)-2-Methoxy-1-(2-(2,2,2-Trifluoroethoxy)Pyridin-4-yl)Ethan-1-Amine hydrochloride

### Step 1: Tert-Butyl (±)-(2-Methoxy-1-(2-(2,2,2-Trifluoroethoxy)Pyridin-4-yl)Ethyl)Carbamate

To a solution of 4-bromo-2-(2,2,2-trifluoroethoxy)pyridine (571 mg, 2.17 mmol, 1.0 eq) in degassed DMSO (100 mL), 2-{[(tert-butoxy)carbonyl]amino}-3-methoxypropanoic acid (749 mg, 3.25 mmol, 1.5 eq), potassium phosphate tribasic (1.41 g, 6.5 mmol, 3.0 eq), 4,4′-di-tert-butyl-2,2′-dipyridyl (59 mg, 0.217 mmol, 0.1 eq), NiCl₂·glyme (49 mg, 0.217 mmol, 0.1 eq) and [lr{dF(CF₃)ppy}₂(dtbpy)]PFₛ (49 mg, 0.04 mmol, 0.02 eq) were added in sequence. The resulting mixture was degassed with N₂ while stirring for 15 minutes. Then, the resulting mixture was stirred at rt overnight under blue LED irradiation. Water was added and the mixture was extracted with EtOAc (3x). The comb. org. layers were further washed with sat. aq. NaCl soln., dried over MgSO₄, filtered and concentrated in vacuo. The residue was purified by Combiflash (column: 24 g, flow: 25 mL/min, Heptane to Heptane + 30% EtOAc) to give an yellow oil. LC-MS (2): t_{R} = 0.98 min; [M+H]⁺: 351.25.

### Step 2: (±)-2-Methoxy-1-(2-(2,2,2-Trifluoroethoxy)Pyridin-4-yl)Ethan-1-Amine Hydrochloride

A solution of tert-butyl (±)-(2-methoxy-1-(2-(2,2,2-trifluoroethoxy)pyridin-4-yl)ethyl)carbamate (600 mg, 1.71 mmol, 1 eq) in 4N HCI in dioxane (6.85 mL, 27.4 mmol, 16 eq) was stirred at rt overnight. The mixture was concentrated in vacuo to afford a white solid. The product was used without further purification. LC-MS (2): t_{R} = 0.56 min; [M+H]⁺: 251.25.

### Synthesis of (±)-1-(2-(Difluoromethoxy)Pyridin-4-yl)Ethan-1-Amine

### Step 1: 1-(2-(Difluoromethoxy)Pyridin-4-yl)Ethan-1-One

To an ice-cooled solution of 2-(difluoromethoxy)pyridine-4-carbonitrile (1.50 g, 8.38 mmol, 1.0 eq) in THF (80 mL), 3 M methylmagnesium bromide solution (6.13 mL, 18.4 mmol, 2.2 eq) was added dropwise. The resulting mixture was stirred at rt overnight. The resulting mixture was quenched with 1 M HCI aq. soln. (15 mL) and the resulting mixture was stirred at rt for 1 h. The reaction mixture was diluted with sat. aq. NaHCO₃ soln. and EtOAc. The layers were separated and the aq. phase was extracted with EtOAc (1× 30 mL). The comb. org. phases were washed with sat. aq. NaCl soln. (1 × 20 mL), dried over MgSO₄, and concentrated in vacuo. The residue was purified by Combiflash (column: 40 g, flow: 40 mL/min, Heptane to Heptane + 18% EtOAc) to give a colorless oil. LC-MS (2): t_{R} = 0.78 min; [M+H]⁺: 188.26.

### Step 2: (±)-1-(2-(Difluoromethoxy)Pyridin-4-yl)Ethan-1-Amine

To a solution of 1-(2-(difluoromethoxy)pyridin-4-yl)ethan-1-one (610 mg, 3.26 mmol, 1 eq) in MeOH (100 mL), ammonium acetate (5.03 g, 65.2 mmol, 20 eq) and sodium cyanoborohydride (410 mg, 6.52 mmol, 2 eq) were added in sequence. The resulting mixture was stirred at rt overnight. The resulting mixture was concentrated in vacuo.The residue was diluted with sat. aq. NaHCO₃ soln. and DCM. The layers were separated and the aq. phase was extracted with DCM (1× 30 mL). The comb. org. phases were washed with sat. aq. NaCl soln. (1 × 20 mL), dried over MgSO₄, and concentrated in vacuo. The residue was used without further purification. LC-MS (2): t_{R} = 0.43 min; [M+H]⁺: 189.31.

### Synthesis of (±)-1-(2-Methyl-6-(2,2,2-Trifluoroethoxy)Pyrimidin-4-yl)Ethan-1-Amine

### Step 1: 2-Methyl-6-(2,2,2-Trifluoroethoxy)Pyrimidine-4-Carbonitrile

To an ice-cooled solution of 2.2,2-trifluoroethanol (1.0 mL, 13.6 mmol, 2.2 eq) in THF (8 mL), sodium hydride 60 % dispersion in mineral oil (569 mg, 14.2 mmol, 2.3 eq) was added portionwise. The resulting mixture was warmed to rt over 30 min and stirred at rt for 30 min. The mixture was cooled to 0° C. and a solution of 6-chloro-2-methylpyrimidine-4-carbonitrile (1.0 g, 6.19 mmol, 1 eq) in THF (4 mL) was added dropwise. The resulting mixture was slowly warmed to rt and stirred at rt for 30 min. The reaction mixture was slowly poured into cold water (40 mL), and then extracted with EtOAc (2× 40 mL). The comb. org. layers were washed with sat. aq. NaCl soln., dried over Na₂SO₄, filtered and concentrated in vacuo. The residue was purified by Combiflash (column: 24 g, flow: 35 mL/min, Heptane to Heptane + 15% TBME) to give a pale yellow oil. LC-MS (2): t_{R} = 0.86 min; [M+H]⁺: 218.30.

### Step 2: 1-(2-Methyl-6-(2,2,2-Trifluoroethoxy)Pyrimidin-4-yl)Ethan-1-One

To a solution cooled at -78° C. of 2-methyl-6-(2,2,2-trifluoroethoxy)pyrimidine-4-carbonitrile (945 mg, 4.35 mmol, 1.0 eq) in THF (37 mL), 3 M methylmagnesium bromide solution in diethyl ether (9.3 mL, 27.8 mmol, 6.4 eq) was added dropwise. The resulting mixture was stirred at rt for 1 hour. The resulting mixture was cooled to 0° C. and slowly quenched with 10% acetic acid aq. soln. (15 mL). The reaction mixture was diluted with sat. aq. NaHCO₃ soln. (50 mL) and EtOAc (50 mL). The layers were separated and the aq. phase was extracted with EtOAc (1× 50 mL). The comb. org. phases were washed with sat. aq. NaCl soln. (1 × 50 mL), dried over Na₂SO₄, and concentrated in vacuo. The residue was purified by Combiflash (column: 40 g, flow: 40 mL/min, Heptane to Heptane + 12% TBME) to give a yellow solid. LC-MS (2): t_{R} = 0.88 min; [M+H]⁺: 235.28.

### Step 3: (±)-1-(2-Methyl-6-(2,2,2-Trifluoroethoxy)Pyrimidin-4-yl)Ethan-1-Amine

To a solution of 1-(2-methyl-6-(2,2,2-trifluoroethoxy)pyrimidin-4-yl)ethan-1-one (271 mg, 1.08 mmol, 1 eq) in MeOH (31 mL), ammonium acetate (1.66 g, 21.5 mmol, 20 eq) and sodium cyanoborohydride (142 mg, 2.15 mmol, 2 eq) were added in sequence. The resulting mixture was stirred at rt overnight. The resulting mixture was concentrated in vacuo. The residue was diluted with sat. aq. NaHCO₃ soln. (40 mL) and DCM (40 mL). The layers were separated and the aq. phase was extracted with DCM (1× 40 mL). The comb. org. phases were washed with sat. aq. NaCl soln. (1 × 40 mL), dried over Na₂SO₄, and concentrated in vacuo. The residue was purified by prep. HPLC (column : Waters XBridge, 30×50 mm, 10 um, UV/MS, basic conditions). LC-MS (4): t_{R} = 0.74 min; [M+H]⁺: 236.06.

### II. Biological Assays

### A) Rat Oscillation Assay Assay Principle

This assay is a functional phenotypic assay designed to mimic epileptic seizures using primary neuronal cultures from embryonic rat brains, which form a functional neuronal network that generate synchronized intracellular calcium concentration oscillations when cultured at high density in 384-well plate. The epileptic phenotype is induced by incubating the neurons in magnesium-free assay buffer, that results in increased probability of NMDA receptor activation, leading to an increased frequency and amplitude of intracellular calcium oscillations. Once neurons are incubated with the calcium indicator dye Fluo-8 AM (Tebu-bio), neuronal calcium oscillations can be monitored in real time using FLIPR® Tetra (fluorometric plate reader, Molecular Devices). With these recordings, the effect of anti-epileptic drugs can be quantified. The anti-epileptic effect of compounds, which activate directly or indirectly the Kv7 channels can be modulated by the Kv7 channel blocker XE-991. The assay was performed as described previously (Pacico N, Mingorance-Le Meur A. New In Vitro Phenotypic Assay for Epilepsy: Fluorescent Measurement of Synchronized Neuronal Calcium Oscillations. PLoS ONE 9(1) 2014) with modifications described hereafter.

### Neuronal Cultures

Animal care followed standard procedures in accordance with swiss institutional guidelines. Dissociated neuronal cultures were obtained from cerebral cortices of embryonic Wistar rats at embryonic stage E18 (Charles River). The uterine horns were removed by caesarian surgery from deeply anesthetized rats (Isofurane) and sacrificed by decapitation. The embryos were decapitated by closing forceps. The brains were isolated and dissected one by one in ice-cold PBS (Life Technologies) under optical control using a binocular. Meninges, olfactory bulbs, and basal ganglia were removed. Cortical hemispheres (still including the hippocampus) were cut in small pieces with tweezers and placed on ice in prechilled Hibernate-E medium (Life technology). The hemispheres were then incubated in 10 mL of Hibernate-E containing 15 U/mL papain (Worthington) for 25 min at 30° C. with gentle mixing every 10 min. Genomic DNA was then digested by prolonging the incubation during 10 min at 37° C. in presence of 4 U/mL rDNase I (Ambion). The obtained suspension was then centrifuged at 800 g for 5 min and the cell pellet was resuspended in 2 mL Hibernate-E and gently dissociated by pipetting up and down 10 times with a plastic Pasteur pipette resulting in a homogenous cell suspension. This suspension was immediately filtered through a 70 µm cell strainer (MACS® SmartStrainer, Miltenyi), collected in 10 mL Hibernate-E and centrifuged at 800 g for 5 min. The cell pellet was resuspended in Neurobasal medium, supplemented with 2% B-27, 0.5 mM Glutamax-I, 100 U/mL penicillin, 100 µg/mL streptomycin (Life Technologies) and diluted at the final concentration of 300′000 cells/mL. One day before plating the cells, 384-well plates were coated with 25 µL/well of 0.1 % poly-L-lysine solution (Sigma), incubated overnight at 37° C., washed two times with sterile distilled water and allowed to dry at room temperature for >2 h. The neurons were seeded at a density of 15′000 cells/well in 50 µL/well in a 384-well black, clear-bottomed plates (Corning) and subsequently maintained in an incubator at 37° C., 5% CO₂ and 95% humidity for 8 to 10 days. After 3 and 7 days, 40% of media was renewed under sterile conditions.

### Protocol Rat Oscillation Assay

Neurons seeded in the assay plates were washed with Hank’s balanced salt solution (HBSS) devoid of Ca²⁺ and Mg²⁺, supplemented with 20 mM HEPES (Life Technologies) and 2 mM CaCl₂ (Sigma), pH 7.4 (hereafter called Assay buffer) using a Biotek EL406 plate washer. Neurons were loaded with 1 µM Fluo-8 AM in Assay buffer for 15 min at 37° C., 5% CO₂. Buffer containing dye was then removed and the assay plates were washed 3 times with Assay buffer using the Biotek EL406 washer and allowed to equilibrate in 50 µL of assay buffer at room temperature for 25 min. The kinetic curves of fluorescence fluctuations acquired once per second using FLIPR® Tetra reflect neuronal calcium oscillations. Recording was performed in two phases separated by 20 min resulting in two acquisitions: “Acute” and “20 min”. In the “Acute” acquisition phase, fluorescence was recorded over a period of 500 sec in presence or absence of the Kv7 channel blocker XE-991. Test compounds were added 250 sec after acquisition start. 20 min after compound addition, calcium oscillations were recorded again for 400 sec, corresponding to the “20 min” acquisition phase.

Stock solutions of test compounds were prepared at a concentration of 10 mM in DMSO (Sigma). 5-fold serial dilutions of the compounds were first prepared in DMSO. Compounds were then diluted in Assay buffer supplemented with 0.1% fatty-acid free bovine serum albumin (Sigma), reaching final compound concentrations of 128 pM to 10 µM on the neurons. The Kv7 channel blocker XE-991 (Biotrend) was directly diluted in Assay buffer containing 0.1% fatty-acid free bovine serum albumin, yielding a final concentration of 10 µM in the assay plate.

### Analysis

Time-sequence data were exported using Screenworks® software (Molecular Devices) and converted with Orbit software (Idorsia Pharmaceuticals ltd.) to a format compatible with proprietary analysis softwares. A high-pass filter was then applied to flatten the signal using HTStudio (Idorsia Pharmaceuticals ltd.) to allow calculations of areas under the curve (AUC) for all time-point and compound concentrations. This allowed to calculate potencies (IC₅₀) at both “Acute” phase and “20min” phase (“IC₅₀acute” and “IC₅₀20 min”) using IC50Studio (Idorsia Pharmaceuticals ltd.) as described hereafter. Note: alternatively, signal flattening and IC₅₀ calculations can be achieved using commercially available softwares such as Igor Pro® from Wave Metric (“moving window” filter) and Prism 7.0 from GraphPad, respectively.
“IC₅₀acute”: the ratio of AUC fluorescence before and after compound addition was used to generate concentration-response curve (inhibition) using non-linear regression analysis with a 4-parameter fitting.
“IC₅₀20 min”: the AUC of fluorescence measured 20 min after compounds addition was used to generate concentration-response curve (inhibition) using non-linear regression analysis with a 4-parameter fitting.

IC₅₀ value corresponds to the compound concentration that inhibits 50% of the neuronal oscillations in the presence of vehicle (top plateau). The maximum of inhibition corresponds to the full abolishment of oscillations (bottom plateau), which was obtained by addition of 100 µM carbamazepine (Sigma). Shift value was calculated as follows: Shift value = (IC₅₀acute value in presence of 10 µM XE-991 [nM]) / (IC₅₀acute value [nM]). If IC₅₀ in presence of XE-991 could not be calculated, then the minimal Shift value was calculated as follows: Shift value= (highest tested concentration [nM]) / (IC₅₀acute value [nM]), and the Shift value was annotated with “>”.

TABLE 1

Rat oscillation IC₅₀s and shift

| Example No | FLIPR: IC₅₀ [nM] | | Shift | Example No | FLIPR: IC₅₀ [nM] | | Shift |
|---|---|---|---|---|---|---|---|
| | acute | 20 min | | | acute | 20 min | |
| 1 | 486 | 855 | >39 | 30 | 804 | 1477 | 4.9 |
| 2 | 291 | 466 | 10 | 31 | 190 | 763 | >96 |
| 3 | 188 | 334 | 18 | 32 | 67 | 303 | 28 |
| 4 | 138 | 1060 | 25 | 33 | 946 | 1511 | >4.9 |
| 5 | 296 | 216 | 8.6 | 34 | 247 | 291 | 13 |
| 6 | 126 | 271 | 39 | 35 | 231 | 717 | 13 |
| 7 | 63 | 100 | 21 | 36 | 245 | 344 | 15 |
| 8 | 168 | 331 | 3.9 | 37 | 26 | 32 | 32 |
| 9 | 766 | 1420 | 14 | 38 | 135 | 390 | 4.1 |
| 10 | 57 | 51 | 49 | 39 | 41 | 68 | 65 |
| 11 | 741 | 1365 | 10 | 40 | 149 | 248 | 29 |
| 12 | 154 | 164 | >36 | 41 | 73 | 117 | 18 |
| 13 | 14 | 41 | 118 | 42 | 1173 | 1915 | 7.1 |
| 14 | 37 | 79 | 119 | 43 | 578 | 967 | 4.8 |
| 15 | 619 | 960 | 8.2 | 44 | 591 | 1350 | 12 |
| 16 | 716 | 848 | 5.6 | 45 | 131 | 701 | 6.7 |
| 17 | 202 | 239 | 16 | 46 | 148 | 257 | 15 |
| 18 | 377 | 1078 | 3.3 | 47 | 118 | 160 | 35 |
| 19 | 50 | 111 | 22 | 48 | 232 | 484 | 24 |
| 20 | 566 | 467 | 15 | 49 | 247 | 628 | 25 |
| 21 | 373 | 942 | 29 | 50 | 68 | 82 | 429 |
| 22 | 134 | 311 | 16 | 51 | 363 | 515 | 4.2 |
| 23 | 75 | 53 | 17 | 52 | 660 | 738 | 4.9 |
| 24 | 208 | 198 | 9.4 | 53 | 168 | 295 | 39 |
| 25 | 235 | 610 | 7.5 | 54 | 218 | 216 | 11 |
| 26 | 295 | 315 | 11 | 55 | 701 | 1163 | 2.4 |
| 27 | 1345 | 1727 | 8.4 | 56 | 123 | 547 | 32 |
| 28 | 117 | 203 | 47 | 57 | 146 | 314 | 13 |
| 29 | 367 | 2150 | >4.6 | 58 | 51 | 105 | 236 |

### C) Kv7.2/3 Assay (Performed at Charles River)

HEK293 cells were stably transfected with the appropriate ion channel cDNA(s) (human KCNQ2 and KCNQ3 genes). Cells were cultured in Dulbecco’s Modified Eagle Medium /Nutrient mixture F-12 (D-MEM/F-12) supplemented with 10% fetal bovine serum, 100 U/mL penicillin G sodium, 100 µg/mL streptomycin sulfate and selection antibiotics. FLIPR Test Procedure: For FLIPR assay, cells were plated in 384-well black clear-bottomed microtiter plates (BD Biocoat Poly-D-Lysine Multiwell Cell Culture Plate) at 15′000 to 30′000 cells per well. Cells were incubated at 37° C. overnight or until cells reached sufficient density in the wells (near confluent monolayer) to use in fluorescence assays. Fluorescence changes triggered by agonist application were recorded using FLIPR® Tetra and displayed with Screenworks® 4.2 software (Molecular Devices). Assays were performed with the FLIPR potassium assay kit (Molecular Devices) according to the manufacturer’s instructions. Dye-loading: Growth media was removed and replaced with 20 µL of dye loading buffer for 60 min at room temperature. FLIPR Recording (agonist mode): Stock solutions of test compounds were prepared at a concentration of 33.3 mM in DMSO. 5 µL of 5x concentrated test, vehicle, or control compounds solutions prepared in the stimulation buffer (K⁺-free buffer with 5 mM TI⁺) were added to each well and fluorescence recording was continued for 5 min. The agonist effect (EC₅₀ and % effect) of test or control compounds on Kv7 channels was determined as follows: Raw data was exported using Screenworks® 4.2 software and the fluorescence traces were analysed using Microsoft Excel (Microsoft Corp., Redmond, WA). The test compounds responses were expressed as % of maximum response of the control compound Flupirtine (Sigma-Aldrich), which was tested at concentrations ranging from 0.03 to 100 µM. Concentration-response data were fitted to a Hill equation. Non-linear least squares fits were made assuming a simple binding model. If appropriate, fits were weighted by the standard deviation. No assumptions about the fit parameters were made; the fit parameters were determined by the algorithm.

TABLE 2

Kv7.2/7.3 activation

| Example No | FLIPR: EC₅₀ (nM) | Example No | FLIPR: EC₅₀ (nM) |
|---|---|---|---|
| 24 | 143 | 47 | 59 |
| 37 | 30 | 53 | 82 |

### III. Pharmacological Experiments

### Formulation and Administration

Compounds were formulated in a 10% polyethylene glycol 400 (PEG 400) / 90% aqueous solution of 0.5 % methylcellulose (MC 0.5%). Firstly drugs are dissolved in PEG 400 and then suspended in MC 0.5 % for oral gavage at Xmg/5 mL/kg (X see table).

### Audiogenic Seizure-Sensitive Mouse Model of Generalized Convulsive Seizures

1. Procedure: Following two days of acclimatisation, auditory seizures are induced in male juvenile DBA/2J mice (22-24 days old; Janvier Labs, France). Each mouse is placed individually in the exposure chamber, an hemispheric acrylic glass dome (diameter: 50 cm) within a sound-attenuated box. The soundattenuated box is equipped with two house lights and a camera system (Fire-I from Unibrain) in order to observe and record the behavioral seizure response. After 60 seconds of habituation, the stimulus, a mixed frequency tone of 15-20 kHz at 110 dB (SASLab Lite, Avisoft Bioacoustics), is played from a speaker that is placed on the top center of the dome. The stimulus is applied for 60 seconds maximum or until the mouse shows tonic extension of the hind limbs. Seizures are classified as following: stage 0, normal behavior; stage 1, wild running; stage 2, generalized clonus; stage 3, tonic extension of the hind limbs.
2. Compounds testing: Acute compound effects on audiogenic generalized convulsive seizures are evaluated in independent groups of 8-10 mice randomly assigned. Following oral administration of compound or vehicle, the maximum seizure stage during sound exposure is assessed. Compounds are given 1 hour before exposure to the stimulus. Each mouse is exposed to the auditory stimulus only once and euthanized afterwards by CO₂ inhalation.

TABLE 3

Efficacies in the AGS mouse model

| Example No | Formulation | Dose and administration | Time of challenge | Seizure stage [% vs. vehicle] |
|---|---|---|---|---|
| 23 | 90%MC0.5% + 10%PEG400 | 30 mg/kg po | 1h | -85 |
| 24 | 90%MC0.5% + 10%PEG400 | 30 mg/kg po | 1h | -100 |
| 37 | 90%MC0.5% + 10%PEG400 | 15 mg/kg po | 1h | -100 |
| 47 | 90%MC0.5% + 10%PEG400 | 10 mg/kg po | 1h | -92 |
| 53 | 90%MC0.5% + 10%PEG400 | 10 mg/kg po | 1h | -78 |

### Amygdala-Kindling Rat Model

1. Procedure: Adult male Wistar rats (Harlan Laboratories, Netherlands, or Charles Rivers, Germany; body weight 300-350 g) were stereotaxically implanted with twisted bipolar plastic-coated stainless steel electrode (MS333-2-BIU 10 mm, Plastics One) into the right basolateral amygdala under isoflurane anesthesia. To place the electrode, trepanations were made in the skull and the electrode was lowered into the right basolateral amygdala (from bregma: anteriorposterior (AP): -2.5 mm, medio-lateral (ML): -3.5 mm, dorso-ventral (DV): -8.6 mm; α=10⁰) and secured to the skull with screws and dental acrylate. After one week of recovery, they were handled daily and habituated over one week to the kindling setup. Kindling procedure: For a kindling session each rat was placed individually into a smooth acrylic plastic, round-bottomed bowl (0 36 cm, height 36 cm, BASi movement-responsive caging system) and its intracranial implanted electrode was connected to the stimulator (STG4008, Multichannel Systems GmbH) and the recording devices (PowerLab 8/35, ADInstruments Ltd) via a cable (335-340/3 (C), Plastics One). For the kindling procedure, each rat was exposed once daily to an electrical stimulation and the behavioral symptoms of the evoked seizure were observed and classified according to the modified Racine scale (stage 0, arrest, wet dog shakes, normal behaviour; stage 1, facial twitches: nose, lips, eyes; stage 2, chewing, head nodding; stage 3, forelimb clonus; stage 4, rearing, falling on forelimbs; stage 5, rearing, falling on side or back, rolling). The electrical stimulus consists of a 1s-train of 50 Hz square-wave biphasic pulses of 1-ms duration at an intensity of 400µA (suprathreshold intensity). The stimulus was applied daily until each rat was fully kindled, i.e. it showed seizures of severity stage 4 and 5 upon electrical stimulation in at least ten consecutive kindling sessions. Data Scoring and analysis. The duration of electroencephalographic seizures (afterdischarge, AD) was recorded using LabChart7 Pro software (ADInstruments Ltd). Simultaneously, videos were recorded to evaluate seizure stage (SS).
2. Compound testing: Acute drug effects were evaluated in groups of 6-8 fully kindled rats in a randomized cross-over design with 48h between drug and vehicle applications. Following oral administration of drug or vehicle, drug testing included determination of the afterdischarge threshold (the minimal stimulation intensity necessary to evoke an afterdischarge (electroencephalographically measured neuronal hyper-synchronous activity with an amplitude 2-times higher than baseline amplitude and a frequency of 2:1 Hz) of at least 3 sec duration) and monitoring electroencephalographic and behavioral correlates of the evoked seizure at ADT (afterdischarge threshold), including AD duration and SS, by a experimenter blind to treatment assignment.

TABLE 4

Efficacies in the rat kindling model

| Example No | Formulation | Dose and administration | Time of challenge | Seizure stage [% vs veh] | AD duration [% vs veh] |
|---|---|---|---|---|---|
| 47 | 90%MC0.5% + 10%PEG400 | 10 mg/kg po | 1 h | -70 | -85 |
| 53 | 90%MC0.5% + 10%PEG400 | 30 mg/kg po | 1 h | -81 | -93 |

1. A compound of Formula (I) 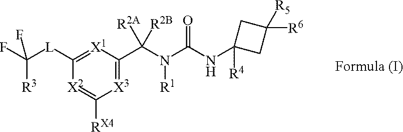
wherein
X¹ represents nitrogen or CR^{X1}; wherein R^{X1} represents hydrogen, halogen, (C₁₋₄)alkyl, or (C₁₋₄)alkoxy;
X² represents nitrogen or CR^{X2}; wherein R^{X2} represents hydrogen, halogen, (C₁₋₄)alkyl, or (C₁₋₄)alkoxy;
X³ represents nitrogen or CR^{X3}; wherein R^{X3} represents hydrogen, halogen, (C₁₋₄)alkyl, (C₁₋₄)alkoxy, or hydroxy;
R¹ represents hydrogen or methyl;
R^{X4} represents hydrogen, halogen, or (C₁₋₄)alkyl;
R^{2A} represents hydrogen; (C₁₋₄)alkyl; (C₂₋₄)alkenyl; (C₂₋₄)alkynyl; (C₃₋₆)cycloalkyl; (C₁₋ ₄)fluoroalkyl; (C₁₋₄)hydroxyalkyl; (C₁₋₄)alkoxy-(C₁₋₂)alkyl; (C₁₋₂)alkoxy-(C₁₋₂)alkoxy-(C₁₋ ₂)alkyl; (C₁₋₂)alkyl-S-(C₁₋₂)alkyl; (C₁₋₂)alkyl-(SO₂)-(C₁₋₂)alkyl; cyano; (C₁₋₂)cyanoalkyl; H₂N-C(O)-(C₁₋₂)alkyl; (R^{N1})₂N-(C₁₋₂)alkyl or (R^{N1})₂N—C(O)—, wherein R^{N1} independently represents hydrogen or (C₁₋₂)alkyl; or a 5-membered heteroaryl group containing one to four nitrogen atoms, wherein said 5-membered heteroaryl group is independently unsubstituted or mono-substituted with (C₁₋₄)alkyl;
and R^{2B} represents hydrogen or methyl; or
R^{2A} and R^{2B} form, together with the carbon atom to which they are attached, a ring of 3- to 6 members, wherein the members needed to complete said ring are each independently selected from —CH₂— and —O— and wherein said ring does not contain more than one —O— member;
L represents a direct bond, cycloprop-1,1-diyl, —CHR^{L}—O—*, —O—CH₂—*, —CH₂—NH—*, —CH₂—N(CH₃)—*, —O—, or —(SO₂)—; wherein R^{L} represents hydrogen, (C₁₋₄)alkyl, CH₃—O—CH₂—, or (CH₃)₂NCH₂-; wherein the asterisks indicate the bond which is linked to the aromatic carbon atom;
R³ represents hydrogen or fluoro;
R⁴ represents hydrogen or (C₁₋₄)alkyl;
R⁵ represents hydrogen, fluoro, or hydroxy; and
R⁶ represents fluoro or (C₁)fluoroalkyl; or
R⁴ and R⁵ together represent a bridge selected from —CH₂— and —CH₂CH₂—; and
R⁶ represents hydrogen, fluoro, (C₁)fluoroalkyl, or (C₁₋₄)alkyl;
or a salt thereof.

2. A compound according to claim 1, wherein
X¹ represents CR^{X1}; wherein R^{X1} represents hydrogen or halogen;
X² represents nitrogen or CH;
X³ represents nitrogen or CH;
R¹ represents hydrogen;
R^{X4} represents hydrogen, halogen, or (C₁₋₄)alkyl;
R^{2A} represents hydrogen; (C₁₋₄)alkyl; (C₁₋₄)fluoroalkyl; (C₁₋₄)hydroxyalkyl; or (C₁₋₄)alkoxy-(C₁₋₂)alkyl;
R^{2B} represents hydrogen;
L represents a direct bond, —CH₂—O—*, or—O—; wherein the asterisk indicates the bond which is linked to the aromatic carbon atom;
R³ represents hydrogen or fluoro;
R⁴ represents hydrogen or (C₁₋₄)alkyl;
R⁵ represents hydrogen, fluoro, or hydroxy; and
R⁶ represents fluoro or (C₁)fluoroalkyl; or
R⁴ and R⁵ together represent a bridge selected from —CH₂— and —CH₂CH₂—; and
R⁶ represents hydrogen, fluoro, (C₁)fluoroalkyl, or (C₁₋₄)alkyl;
or a salt thereof.

3. A compound according to claim 2, wherein R^{2A} represents hydrogen, (C₁₋₄)alkyl, (C₁₋₄)fluoroalkyl, (C₁₋₄)hydroxyalkyl, or methoxymethyl; and R^{2B} represents hydrogen;
or a salt thereof.

4. A compound according to claim 3, wherein L represents a direct bond;
or a salt thereof.

5. A compound according to claim 4, wherein R³ represents fluoro;
or a salt thereof.

6. A compound according to claim 5, wherein R^{X4} represents hydrogen;
or a salt thereof.

7. A compound according to claim 6, wherein each of X¹, X², and X³ represents CH;
or a salt thereof.

8. A compound according to claim 3, wherein the fragment 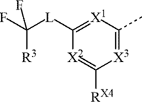
represents: 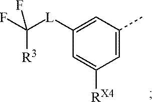
wherein R^{X4} represents hydrogen or halogen; R³ represents hydrogen or fluoro; and L represents a direct bond, —CH₂—O—*, or—O—; wherein the asterisk indicates the bond which is linked to the aromatic carbon atom; or 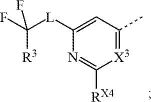
wherein X³ represents nitrogen or CH; R^{X4} represents hydrogen or (C₁₋₄)alkyl; R³ represents hydrogen or fluoro; and L represents —CH₂—O—*, or—O—; wherein the asterisk indicates the bond which is linked to the aromatic carbon atom;
or a salt thereof.

9. A compound according to claim 7, wherein R⁴ represents hydrogen; R⁵ represents hydrogen or fluoro; and R⁶ represents fluoro, difluoromethyl or trifluoromethyl;
or a salt thereof.

10. A compound according to claim 7, wherein R⁴ and R⁵ together represent a —CH₂— bridge; and R⁶ represents hydrogen, fluoro, difluoromethyl, or trifluoromethyl;
or a salt thereof.

11. A compound according to claim 1 selected from the group consisting of: 1-(3,3-Difluoro-cyclobutyl)-3-(3-trifluoromethyl-benzyl)-urea;
1-Bicyclo[1.1.1]pent-1-yl-3-[1-(3-trifluoromethyl-phenyl)-ethyl]-urea;
1-(3-Difluoromethyl-cyclobutyl)-3-[1-(3-trifluoromethyl-phenyl)-ethyl]-urea;
1-(3-Fluoro-bicyclo[1.1.1]pent-1-yl)-3-[1-(3-trifluoromethyl-phenyl)-ethyl]-urea;
1-(3-Difluoromethyl-bicyclo[1.1.1]pent-1-yl)-3-[1-(3-trifluoromethyl-phenyl)-ethyl]-urea;
1-Bicyclo[1.1.1]pent-1-yl-3-[1-(3-trifluoromethoxy-phenyl)-ethyl]-urea;
1-(3-Difluoromethyl-cyclobutyl)-3-[1-(3-trifluoromethoxy-phenyl)-ethyl]-urea;
1-(3-Fluoro-bicyclo[1.1.1]pent-1-yl)-3-[1-(3-trifluoromethoxy-phenyl)-ethyl]-urea;
1-[2,2-Difluoro-1-(3-trifluoromethyl-phenyl)-ethyl]-3-(3-hydroxy-3-trifluoromethyl-cyclobutyl)-urea;
1-(3,3-Difluoro-1-methyl-cyclobutyl)-3-[2,2-difluoro-1-(3-trifluoromethyl-phenyl)-ethyl]-urea;
1-Bicyclo[1.1.1]pent-1-yl-3-[1-(3-difluoromethoxy-phenyl)-ethyl]-urea;
1-(3-Difluoromethyl-bicyclo[1.1.1]pent-1-yl)-3-[2-hydroxy-1-(3-trifluoromethyl-phenyl)-ethyl]-urea;
1-(3-Difluoromethyl-bicyclo[1.1.1]pent-1-yl)-3-[2,2-difluoro-1-(3-trifluoromethyl-phenyl)-ethyl]-urea;
1-(3-Difluoromethyl-bicyclo[1.1.1]pent-1-yl)-3-[2-methoxy-1-(3-trifluoromethyl-phenyl)-ethyl]-urea;
1-(3-Difluoromethyl-bicyclo[1.1.1]pent-1-yl)-3-(2-fluoro-3-trifluoromethyl-benzyl)-urea;
1-(3-Difluoromethyl-bicyclo[1.1.1]pent-1-yl)-3-(3-fluoro-5-trifluoromethyl-benzyl)-urea;
1-(3-Fluoro-bicyclo[1.1.1]pent-1-yl)-3-[3-(2,2,2-trifluoro-ethoxy)-benzyl]-urea;
1-(3-Difluoromethyl-cyclobutyl)-3-[3-(2,2,2-trifluoro-ethoxy)-benzyl]-urea;
1-[3-(2,2,2-Trifluoro-ethoxy)-benzyl]-3-(3-trifluoromethyl-bicyclo[1.1.1]pent-1-yl)-urea;
1-(3-Difluoromethoxy-benzyl)-3-(3-fluoro-bicyclo[1.1.1]pent-1-yl)-urea;
1-(3-Difluoromethoxy-benzyl)-3-(3-difluoromethyl-cyclobutyl)-urea;
1-(3-Difluoromethoxy-benzyl)-3-(3-trifluoromethyl-bicyclo[1.1.1]pent-1-yl)-urea;
1-(3-Trifluoromethoxy-benzyl)-3-(3-trifluoromethyl-bicyclo[1.1.1]pent-1-yl)-urea;
1-(3-Fluoro-bicyclo[1.1.1]pent-1-yl)-3-(3-trifluoromethoxy-benzyl)-urea;
1-(3-Difluoromethyl-cyclobutyl)-3-(3-trifluoromethoxy-benzyl)-urea;
1-Bicyclo[1.1.1]pent-1-yl-3-(3-trifluoromethoxy-benzyl)-urea;
1-(3-Difluoromethyl-benzyl)-3-(3-difluoromethyl-cyclobutyl)-urea;
1-(3-Difluoromethyl-benzyl)-3-(3-trifluoromethyl-bicyclo[1.1.1]pent-1-yl)-urea;
1-(3-Difluoromethyl-cyclobutyl)-3-(2-trifluoromethoxy-pyridin-4-ylmethyl)-urea;
1-(2-Trifluoromethoxy-pyridin-4-ylmethyl)-3-(3-trifluoromethyl-bicyclo[1.1.1]pent-1-yl)-urea;
1-(3-Difluoromethyl-cyclobutyl)-3-{2-methoxy-1-[2-(2,2,2-trifluoro-ethoxy)-pyridin-4-yl]-ethyl}-urea;
1-{2-Methoxy-1-[2-(2,2, 2-trifluoro-ethoxy)-pyrid in-4-yl]-ethyl}-3-(3-trifluorom ethyl-cyclobutyl)-urea;
1-[1-(2-Difluoromethoxy-pyridin-4-yl)-ethyl]-3-(3-difluoromethyl-cyclobutyl)-urea;
1-{1-[2-Methyl-6-(2,2,2-trifluoro-ethoxy)-pyrimidin-4-yl]-ethyl}-3-(3-trifluoromethyl-bicyclo[1.1.1]pent-1-yl)-urea;
1-Bicyclo[1.1.1]pent-1-yl-3-(3-trifluoromethyl-benzyl)-urea;
1-(3-Fluoro-bicyclo[1.1.1]pent-1-yl)-3-(3-trifluoromethyl-benzyl)-urea;
1-(3-Trifluoromethyl-benzyl)-3-(3-trifluoromethyl-bicyclo[1.1.1]pent-1-yl)-urea;
1-(3-Difluoromethyl-cyclobutyl)-3-(3-trifluoromethyl-benzyl)-urea;
1-(3-Methyl-bicyclo[1.1.1]pent-1-yl)-3-(3-trifluoromethyl-benzyl)-urea;
1-(3-Fluoromethyl-bicyclo[1.1.1]pent-1-yl)-3-(3-trifluoromethyl-benzyl)-urea;
1-(3-Trifluoromethyl-benzyl)-3-(3-trifluoromethyl-cyclobutyl)-urea;
1-(3-Hydroxy-3-trifluoromethyl-cyclobutyl)-3-(3-trifluoromethyl-benzyl)-urea;
1-Bicyclo[1.1.1]pent-1-yl-3-[2-(2,2,2-trifluoro-ethoxy)-pyridin-4-ylmethyl]-urea;
1-(3-Fluoro-bicyclo[1.1.1]pent-1-yl)-3-[2-(2,2,2-trifluoro-ethoxy)-pyridin-4-ylmethyl]-urea;
1-[2-(2,2,2-Trifluoro-ethoxy)-pyridin-4-ylmethyl]-3-(3-trifluoromethyl-bicyclo[1.1.1]pent-1-yl)-urea;
1-(3-Difluoromethyl-cyclobutyl)-3-[2-(2,2,2-trifluoro-ethoxy)-pyridin-4-ylmethyl]-urea;
1-(3-Difluoromethyl-bicyclo[1.1.1]pent-1-yl)-3-(3-trifluoromethyl-benzyl)-urea;
1-Bicyclo[2.1.1]hex-1-yl-3-(3-trifluoromethyl-benzyl)-urea;
1-(3,3-Difluoro-1-methyl-cyclobutyl)-3-(3-trifluoromethyl-benzyl)-urea;
1-(3-(trifluoromethyl)benzyl)-3-((1s,3s)-3-(trifluoromethyl)cyclobutyl)urea;
1-(3-(trifluoromethyl)benzyl)-3-((1r,3r)-3-(trifluoromethyl)cyclobutyl)urea;
1-((1s,3s)-3-(difluoromethyl)cyclobutyl)-3-(3-(trifluoromethyl)benzyl)urea;
1-((1r,3r)-3-(difluoromethyl)cyclobutyl)-3-(3-(trifluoromethyl)benzyl)urea;
1-{(S)-1-[2-Methyl-6-(2,2,2-trifluoro-ethoxy)-pyrimidin-4-yl]-ethyl}-3-(3-trifluoromethyl-bicyclo[1.1.1]pent-1-yl)-urea;
1-((1r,3r)-3-(difluoromethyl)cyclobutyl)-3-((2-(2,2,2-trifluoroethoxy)pyridin-4-yl)methyl)urea;
1-((1s,3s)-3-(difluoromethyl)cyclobutyl)-3-((2-(2,2,2-trifluoroethoxy)pyridin-4-yl)methyl)urea;
1-((1s,3R)-3-(difluoromethyl)cyclobutyl)-3-((S)-1-(3-(trifluoromethoxy)phenyl)ethyl)urea; and
1-((1r,3S)-3-(difluoromethyl)cyclobutyl)-3-((S)-1-(3-(trifluoromethoxy)phenyl)ethyl)urea;
or a salt thereof.

12. A pharmaceutical composition comprising, as active principle, a compound according to claim 1, or a pharmaceutically acceptable salt thereof, and at least one therapeutically inert excipient.

13. (canceled)

14. A method for prevention or treatment of a disease selected from epilepsy, myokymia, tinnitus, hearing disorders, neuropathic and inflammatory pain, psychiatric disorders, substance use disorders, neurological disorders, and diseases affecting the smooth muscles in a patient, wherein the method comprises administering to the patient a pharmaceutically active amount of a compound according to claim 1 or a pharmaceutically acceptable salt thereof.

15. (canceled)

16. A compound according to claim 8, wherein R⁴ represents hydrogen; R⁵ represents hydrogen or fluoro; and R⁶ represents fluoro, difluoromethyl or trifluoromethyl;
or a salt thereof.

17. A compound according to claim 8, wherein R⁴ and R⁵ together represent a —CH₂— bridge; and R⁶ represents hydrogen, fluoro, difluoromethyl, or trifluoromethyl;
or a salt thereof.

18. A compound according to claim 7, wherein R¹ represents hydrogen; or a salt thereof.

19. A compound according to claim 9, wherein R¹ represents hydrogen; or a salt thereof.

20. A compound according to claim 10, wherein R¹ represents hydrogen; or a salt thereof.

21. A compound according to claim 1, wherein the compound is 1-(3-difluoromethyl-bicyclo[1.1.1]pent-1-yl)-3-(3-trifluoromethyl-benzyl)-urea.

22. A compound according to claim 1, wherein the compound is 1-(3-(difluoromethyl)cyclobutyl)-3-(3-(trifluoromethyl)benzyl)urea.

23. A pharmaceutical composition comprising, as active principle, the compound according to claim 21 and at least one therapeutically inert excipient.

24. A pharmaceutical composition comprising, as active principle, the compound according to claim 22 and at least one therapeutically inert excipient.

25. A method for prevention or treatment of a disease selected from epilepsy, myokymia, tinnitus, hearing disorders, neuropathic and inflammatory pain, psychiatric disorders, substance use disorders, neurological disorders, and diseases affecting the smooth muscles in a patient, wherein the method comprises administering to the patient a pharmaceutically active amount of the compound according to claim 21.

26. A method for prevention or treatment of a disease selected from epilepsy, myokymia, tinnitus, hearing disorders, neuropathic and inflammatory pain, psychiatric disorders, substance use disorders, neurological disorders, and diseases affecting the smooth muscles in a patient, wherein the method comprises administering to the patient a pharmaceutically active amount of the compound according to claim 22.
